(19) United States
(12) Patent Application Publication
(10) Pub. No.: US 2010/0280050 A1
(43) Pub. Date: Nov. 4, 2010

(54) Piperidinylamino-Thieno[2,3-D] Pyrimidine Compounds for Treating Fibrosis

(76) Inventors: Kimberley Gannon, Watertown, MA (US); Ohilip B. Graham, Carlisle, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/676,454

(22) PCT Filed: Sep. 4, 2008
(86) PCT No.: PCT/US08/75193

§ 371 Date: Jul. 6, 2010

Related U.S. Application Data
(60) Provisional application No. 60/969,820, filed on Sep. 4, 2007.

(51) Int. Cl. A61K 31/519; A61P 43/00

(52) U.S. Cl. 514/260.1

(57) ABSTRACT

The invention provides a method of treating or preventing fibrosis in a subject by administering a 5-HT modulator, e.g., a 5-HT_{2B} modulator. In particular embodiments, the 5-HT modulator is a piperidinylamino-thieno[2,3-d]pyrimidine compound.

### RELATED APPLICATIONS

This application claims the benefit of priority to U.S. Provisional Patent Application Ser. No. 60/969,820, filed Sep. 4, 2007, the contents of which are hereby incorporated by reference.

### FIELD OF THE INVENTION

The invention generally relates to the field of serotonin (5-hydroxytryptamine, or 5-HT) receptor modulators, e.g., antagonists, and more particularly to piperidinylamino-thieno[2,3-d]pyrimidine compounds which are 5-HT modulators, and use of these compounds in the treatment and/or prevention of fibrosis.

### BACKGROUND OF THE INVENTION

Fibrosis is characterized by the abnormal accumulation of fibrous tissue. Fibrous tissue accumulates naturally as part of the physiological process of repairing damaged bodily tissue. However, abnormal accumulations of fibrous tissue can be harmful to bodily organs, impairing proper functioning of the organ. For example, abnormal accumulation of fibrous tissue in the liver, lung, and kidney can impair proper functioning of these organs.

Liver fibrosis occurs as a part of the wound-healing response to chronic liver injury. Liver injuries leading to fibrosis can be caused by parasitic infection, trauma, and autoimmune diseases. Parasitic infections causing liver fibrosis can be due to either extracellular parasites (e.g., Shistosomes, Clonochis, Fasciola, Opisthorchis, and Dicrocoelium) or intracellular parasites (e.g., fungi and certain bacteria). Haemochromatosis, Wilson's disease, alcoholism, schistosomiasis, bile duct obstruction, exposure to toxins, metabolic disorders, certain bacterial infections, sepsis, hypoxia, non-alcoholic fatty liver disease, non-alcoholic steatohepatitis, and exposure to certain medications can also lead to liver fibrosis.

Another cause of liver fibrosis is viral infection. For example, hepatitis A, B, and C, hepatitis delta and epsilon virus, and other viruses that are trophic for hepatic cells can cause liver fibrosis. The hepatitis C virus (HCV) is a major cause of liver fibrosis. It is estimated that hepatitis C virus affects about 170 million people worldwide, including 5 million in Western Europe and 2.7-4 million people in the United States (Vrolijk et al. (2004) Netherlands J. Med. 62:76-82; Saadeh and Davis (2004) Cleveland Clinic J. of Med. 71:S3-S7; Foster (2003) Expert Opin. Pharmacother. 4:685-691). The prevalence of HCV varies from 0.5%-2% in most developed countries but is as high as 20% in Egypt (Foster, supra). Some reports indicate that about 70-80% of those infected by HCV develop chronic infections, of which about one-quarter are at risk of developing severe fibrosis within 20 years and half within 50 years. The remaining half of chronically infected individuals remain relatively asymptomatic (Schuppan et al. (2003) Cell Death and Differentiation 10:S59-S67; Patel and McHutchison (2003) Chronic Hepatitis C 114:48-62).

Therapeutic methods for treating or preventing liver fibrosis are important because fibrosis of the liver can result in cirrhosis, liver failure, and even death. Moreover, current therapeutic methods for treating liver fibrosis, which include removal of the underlying cause, e.g., toxin or infectious agent, suppression of inflammation using corticosteroids or IL-1 receptor antagonists, and down-regulation of stellate cell activation using gamma interferon or antioxidants, each suffer drawbacks.

Kidney fibrosis can occur in response to a variety of conditions, including hypertension and as a side effect to certain medications. Fibrosis of the kidney impairs renal function and can lead to chronic renal failure, a gradual and progressive loss of the ability of the kidneys to excrete wastes, concentrate urine, and conserve electrolytes. However, treatment options for this debilitating disease are limited.

Accordingly, the need remains for new methods and compositions for treating fibrosis.

### SUMMARY OF THE INVENTION

The present invention relates in part to a method of treating or preventing fibrosis in a subject by administering a 5-HT modulator, e.g., a 5-HT_{2B} modulator. In particular embodiments, the 5-HT modulator is a piperidinylamino-thieno[2,3-d]pyrimidine compound. For example, one aspect of the invention relates to a method of treating or preventing fibrosis of an organ of a subject, comprising administering to a subject in need thereof a therapeutically effective amount of a compound of formula I or a composition comprising a therapeutically effective amount of a compound of formula I, wherein formula I is represented by:

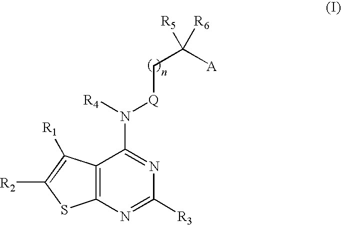

including pharmaceutically acceptable salts, solvates, and/or esters thereof; wherein

R₁ and R₂ represent independently hydrogen, lower alkyl, C₁-C₆ cycloalkyl or cycloheteroalkyl, halogen, halo-substituted alkyl, —COOH, —CN, —NH₂, —NO₂, —OH, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or

R₁ and R₂, taken together with their bonded carbon atoms, form a substituted or unsubstituted C₄-C₇ cycloalkyl or cycloheteroalkyl; wherein the C₄-C₇ cycloheteroalkyl comprises at least one of O, N or S, and the substituted C₄-C₇ cycloalkyl or cycloheteroalkyl comprises at least one substituent selected from halogen, —COOH, —CN, —NH₂, —NO₂, —OH, lower alkyl, substituted lower alkyl, substituted or unsubstituted C₁-C₆ cycloalkyl or cycloheteroalkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, and —R₉-haloalkoxy;

R₃ is H, halogen, —CN, —NH₂, lower alkyl, R₇, —OR₇, —NHR₇, —N(R₇)₂, or substituted or unsubstituted aryl or heteroaryl;

R₄ is H, R₇, or substituted or unsubstituted aryl or heteroaryl;

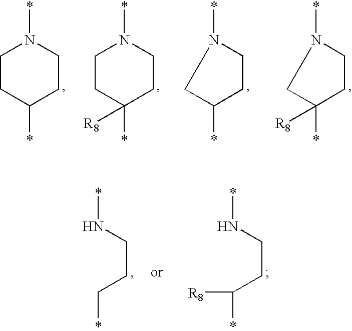

Q is

R₅ and R₆ represent independently hydrogen, halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or

R₅, R₆, and A taken together with their bonded carbons, form a substituted or unsubstituted unsaturated 5- or 6-membered carbocyclic ring or a substituted or unsubstituted saturated 5-, 6-, or 7-membered carbocyclic ring, wherein the carbocyclic ring may be a fused biaryl ring or a heterocarbocyclic ring comprising at least one heteroatom selected from the group consisting of O, N, S and P; and the substituted ring comprises at least one of halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or R₅, R₆, and A, taken together with their bonded carbons, form an aromatic ring that is optionally substituted on the adjacent carbon atoms to form a bicyclic ring with a 5- or 6-membered unsaturated or saturated ring;

R₇ represents independently for each occurrence substituted or unsubstituted C₁-C₆ alkyl or C₃-C₆ cycloalkyl or C₃-C₆ cycloheteroalkyl;

R₈ is hydrogen, halogen, CN, or a substituted or unsubstituted lower alkyl;

R₉ represents independently for each occurrence substituted or unsubstituted C₁-C₆ alkylene or C₃-C₆ cycloalkylene or C₃-C₆ cycloheteroalkylene;

A is hydrogen or C₁-C₆ alkyl;

n is 0, 1, 2, 3, 4 or 5; and

* represents a point of attachment.

In an embodiment, R₁ and R₂ represent independently hydrogen, lower alkyl, or halogen. In an embodiment, R₃ and R₄ represent independently hydrogen or unsubstituted C₁-C₆ alkyl. In an embodiment Q is

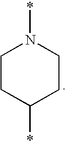

In an embodiment, R₅ is substituted aryl; R₆ is hydrogen; and A is H. In an embodiment, n is 0 or 1.

In an embodiment, R₅, R₆, and A, taken together with their bonded carbons, form an aromatic ring, e.g., phenyl, naphthyl, diphenylmethyl, biaryl; that is optionally substituted on the adjacent carbon atoms to form a bicyclic ring with a 5- or 6-membered unsaturated or saturated ring such as

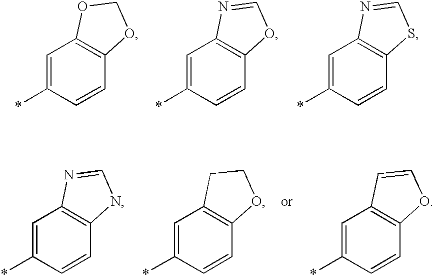

In an embodiment, R₁ is H, —CH₃, —CH(CH₃)₂, or Cl. In another embodiment, R₂ is H, Cl, lower alkyl, e.g., straight or branched C₁, C₂, C₃ (e.g., iso- or tert-butyl), C₄ or C₅ alkyl, or aryl, e.g., phenyl or fluorophenyl. R₁ and R₂ may also, taken together with the bonded carbons from the thieno, form a cyclohexyl ring. The Q group is preferably an N-substituted alkyl or cycloalkyl. The linking group denoted by ( )ₙ may be substituted or unsubstituted, straight or branched, and may be a single bond, or made up of 1, 2, 3, 4 or 5 carbons or more. In certain embodiments, n is 2, 3, 4 or 5. In certain embodiments, A is H or —CH₃. In certain embodiments, A is H.

In an embodiment, the compound has the following formula:

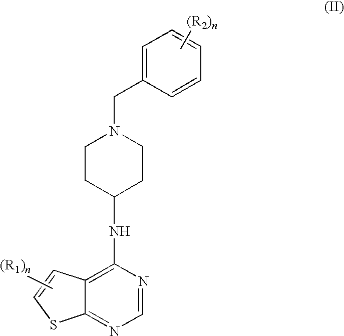

including pharmaceutically acceptable salts, solvates, and/or esters thereof; wherein R₁ represents independently for each occurrence halogen, lower alkyl, cyano, or trihalomethyl; R₂ represents independently for each occurrence hydrogen, halogen, cyano, trihalomethyl, lower alkoxy, carboxylate, amide, or a sulfonyl group; and n represents independently for each occurrence 1 or 2.

In an embodiment, when n is 1, R₂ is not hydrogen, and when n is 2, both R₂ groups are not hydrogen. Examples of amides include amido, N-methylamido and dimethylamido groups; examples of sulfonyl groups include trifluoromethylsulfonyl, sulfonyl, and methylsulfonyl groups. In certain embodiments, the pharmaceutically acceptable salt is a maleate, hydrochloride, or fumarate salt.

In an embodiment, the compound has the following formula:

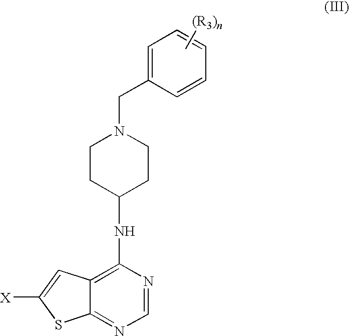

including pharmaceutically acceptable salts, solvates, and/or esters thereof; wherein, X is halogen; R₃ represents independently for each occurrence halogen, cyano, or trihalomethyl; and n is 1 or 2. In certain embodiments, the pharmaceutically acceptable salt is a maleate, hydrochloride, or fumarate salt.

In an embodiment, the compound is 5-((4-(6-chlorothieno[2,3-d]pyrimidin-4-ylamino)piperidin-1-yl)methyl)-2-fluorobenzonitrile or a pharmaceutically acceptable salt thereof.

In an embodiment, the compound is 3-((4-(6-chlorothieno[2,3-d]pyrimidin-4-ylamino)piperidin-1-yl)methyl)benzonitrile or a pharmaceutically acceptable salt thereof.

In an embodiment, the organ is the liver. In an embodiment, the organ is the kidney. In an embodiment, the organ is the lung. In an embodiment, the subject is a mammal. In an embodiment, the subject is a human. The compound may be administered in dosages as to be determined by one of skill in the art or as described herein. In an embodiment, the compound of formula I is administered at a dosage in the range of about 20 mg to about 1000 mg. In an embodiment, the mode of administration of said compound is oral, intravenous, sublingual, ocular, transdermal, rectal, topical, intramuscular, intra-arterial, subcutaneous, buccal, nasal, or direct delivery to the liver.

Another aspect of the invention relates to a method of treating or preventing necrosis or inflammation in a subject, comprising administering to a subject in need thereof a therapeutically effective amount of a compound of formula I or a composition comprising a therapeutically effective amount of a compound of formula I, wherein formula I is represented by:

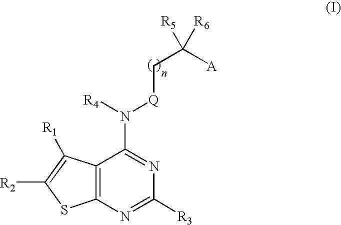

including pharmaceutically acceptable salts, solvates, and/or esters thereof; wherein

R₁ and R₂ represent independently hydrogen, lower alkyl, C₁-C₆ cycloalkyl or cycloheteroalkyl, halogen, halo-substituted alkyl, —COOH, —CN, —NH₂, —NO₂, —OH, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or

R₁ and R₂, taken together with their bonded carbon atoms, form a substituted or unsubstituted C₄-C₇ cycloalkyl or cycloheteroalkyl; wherein the C₄-C₇ cycloheteroalkyl comprises at least one of O, N or S, and the substituted C₄-C₇ cycloalkyl or cycloheteroalkyl comprises at least one substituent selected from halogen, —COOH, —CN, —NH₂, —NO₂, —OH, lower alkyl, substituted lower alkyl, substituted or unsubstituted C₁-C₆ cycloalkyl or cycloheteroalkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, and —R₉-haloalkoxy;

R₃ is H, halogen, —CN, —NH₂, lower alkyl, R₇, —OR₇, —NHR₇, —N(R₇)₂, or subst unsubstituted aryl or heteroaryl;

R₄ is H, R₇, or substituted or unsubstituted aryl or heteroaryl;

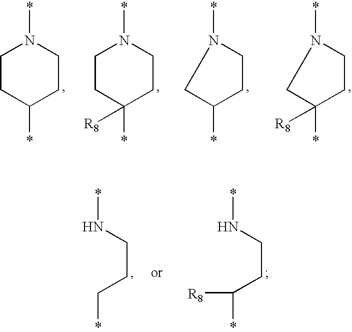

Q is

R₅ and R₆ represent independently hydrogen, halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or

R₅, R₆, and A taken together with their bonded carbons, form a substituted or unsubstituted unsaturated 5- or 6-membered carbocyclic ring or a substituted or unsubstituted saturated 5-, 6-, or 7-membered carbocyclic ring, wherein the carbocyclic ring may be a fused biaryl ring or a heterocarbocyclic ring comprising at least one heteroatom selected from the group consisting of O, N, S and P; and the substituted ring comprises at least one of halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or R₅, R₆, and A, taken together with their bonded carbons, form an aromatic ring that is optionally substituted on the adjacent carbon atoms to form a bicyclic ring with a 5- or 6-membered unsaturated or saturated ring;

R₇ represents independently for each occurrence substituted or unsubstituted C₁-C₆ alkyl or C₃-C₆ cycloalkyl or C₃-C₆ cycloheteroalkyl;

R₈ is hydrogen, halogen, CN, or a substituted or unsubstituted lower alkyl;

R₉ represents independently for each occurrence substituted or unsubstituted C₁-C₆ alkylene or C₃-C₆ cycloalkylene or C₃-C₆ cycloheteroalkylene;

A is hydrogen or C₁-C₆ alkyl;

n is 0, 1, 2, 3, 4 or 5; and

* represents a point of attachment.

In an embodiment, R₁ and R₂ represent independently hydrogen, lower alkyl, or halogen. In an embodiment, R₃ and R₄ represent independently hydrogen or unsubstituted C₁-C₆ alkyl. In an embodiment, Q is

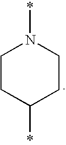

In an embodiment, R₅ is substituted aryl; R₆ is hydrogen; and A is H. In an embodiment, n is 0 or 1.

In an embodiment, R₅, R₆, and A, taken together with their bonded carbons, form an aromatic ring, e.g., phenyl, naphthyl, diphenylmethyl, biaryl; that is optionally substituted on the adjacent carbon atoms to form a bicyclic ring with a 5- or 6-membered unsaturated or saturated ring such as

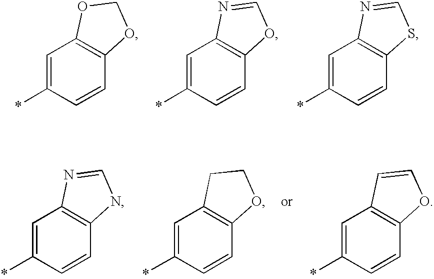

In an embodiment, R₁ is H, —CH₃, —CH(CH₃)₂, or Cl. In another embodiment, R₂ is H, Cl, lower alkyl, e.g., straight or branched C₁, C₂, C₃ (e.g., iso- or tert-butyl), C₄ or C₅ alkyl, or aryl, e.g., phenyl or fluorophenyl. R₁ and R₂ may also, taken together with the bonded carbons from the thieno, form a cyclohexyl ring. The Q group is preferably an N-substituted alkyl or cycloalkyl. The linking group denoted by ( )ₙ may be substituted or unsubstituted, straight or branched, and may be a single bond, or made up of 1, 2, 3, 4 or 5 carbons or more. In certain embodiments, n is 2, 3, 4 or 5. In certain embodiments, A is H or —CH₃. In certain embodiments, A is H.

In an embodiment, the compound has the following formula:

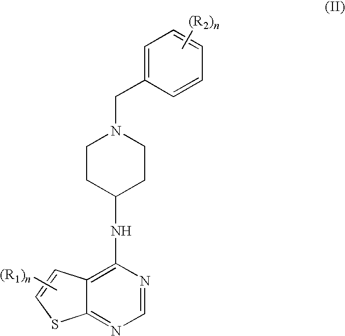

including pharmaceutically acceptable salts, solvates, and/or esters thereof; wherein R₁ represents independently for each occurrence halogen, lower alkyl, cyano, or trihalomethyl; R₂ represents independently for each occurrence hydrogen, halogen, cyano, trihalomethyl, lower alkoxy, carboxylate, amide, or a sulfonyl group; and n represents independently for each occurrence 1 or 2.

In an embodiment, when n is 1, R₂ is not hydrogen, and when n is 2, both R₂ groups are not hydrogen. Examples of amides include amido, N-methylamido and dimethylamido groups; examples of sulfonyl groups include trifluoromethylsulfonyl, sulfonyl, and methylsulfonyl groups. In certain embodiments, the pharmaceutically acceptable salt is a maleate, hydrochloride, or fumarate salt.

In an embodiment, the compound has the following formula:

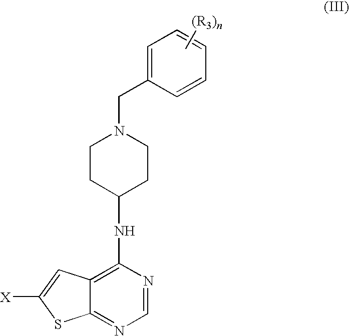

including pharmaceutically acceptable salts, solvates, and/or esters thereof; wherein, X is halogen; R₃ represents independently for each occurrence halogen, cyano, or trihalomethyl; and n is 1 or 2. In certain embodiments, the pharmaceutically acceptable salt is a maleate, hydrochloride, or fumarate salt.

In an embodiment, the compound is 5-((4-(6-chlorothieno[2,3-d]pyrimidin-4-ylamino)piperidin-1-yl)methyl)-2-fluorobenzonitrile or a pharmaceutically acceptable salt thereof.

In an embodiment, the compound is 3-((4-(6-chlorothieno[2,3-d]pyrimidin-4-ylamino)piperidin-1-yl)methyl)benzonitrile or a pharmaceutically acceptable salt thereof.

In an embodiment, the subject is a mammal. In an embodiment, the subject is a human. The compound may be administered in dosages as to be determined by one of skill in the art or as described herein. In an embodiment, the compound of formula I is administered at a dosage in the range of about 20 mg to about 1000 mg. In an embodiment, the mode of administration of said compound is oral, intravenous, sublingual, ocular, transdermal, rectal, topical, intramuscular, intra-arterial, subcutaneous, buccal, nasal, or direct delivery to the liver. In an embodiment, the necrosis is associated with a viral, bacterial, or parasitic infection. In an embodiment, the necrosis results from exposure of the subject to a toxin or therapeutic agent. In an embodiment, the inflammation is associated with a viral, bacterial, or parasitic infection. In an embodiment, the inflammation results from exposure of the subject to a toxin or therapeutic agent.

Another aspect of the invention relates to a method of inducing apoptosis of activated hepatic stellate cells in a subject, comprising administering to a subject an effective amount of a compound of formula I or a composition comprising a therapeutically effective amount of a compound of formula I, wherein formula I is represented by:

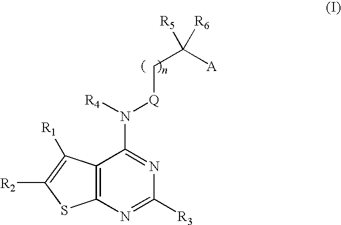

including pharmaceutically acceptable salts, solvates, and/or esters thereof; wherein

R₁ and R₂ represent independently hydrogen, lower alkyl, C₁-C₆ cycloalkyl or cycloheteroalkyl, halogen, halo-substituted alkyl, —COOH, —CN, —NH₂, —NO₂, —OH, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or

R₁ and R₂, taken together with their bonded carbon atoms, form a substituted or unsubstituted C₄-C₇ cycloalkyl or cycloheteroalkyl; wherein the C₄-C₇ cycloheteroalkyl comprises at least one of O, N or S, and the substituted C₄-C₇ cycloalkyl or cycloheteroalkyl comprises at least one substituent selected from halogen, —COOH, —CN, —NH₂, —NO₂, —OH, lower alkyl, substituted lower alkyl, substituted or unsubstituted C₁-C₆ cycloalkyl or cycloheteroalkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, and —R₉-haloalkoxy;

R₃ is H, halogen, —CN, —NH₂, lower alkyl, R₇, —OR₇, —NHR₇, —N(R₇)₂, or substituted or unsubstituted aryl or heteroaryl;

R₄ is H, R₇, or substituted or unsubstituted aryl or heteroaryl;

Q is

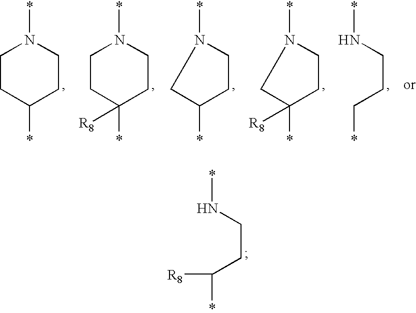

R₅ and R₆ represent independently hydrogen, halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or

R₅, R₆, and A taken together with their bonded carbons, form a substituted or unsubstituted unsaturated 5- or 6-membered carbocyclic ring or a substituted or unsubstituted saturated 5-, 6-, or 7-membered carbocyclic ring, wherein the carbocyclic ring may be a fused biaryl ring or a heterocarbocyclic ring comprising at least one heteroatom selected from the group consisting of O, N, S and P; and the substituted ring comprises at least one of halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or R₅, R₆, and A, taken together with their bonded carbons, form an aromatic ring that is optionally substituted on the adjacent carbon atoms to form a bicyclic ring with a 5- or 6-membered unsaturated or saturated ring;

R₇ represents independently for each occurrence substituted or unsubstituted C₁-C₆ alkyl or C₃-C₆ cycloalkyl or C₃-C₆ cycloheteroalkyl;

R₈ is hydrogen, halogen, CN, or a substituted or unsubstituted lower alkyl;

R₉ represents independently for each occurrence substituted or unsubstituted C₁-C₆ alkylene or C₃-C₆ cycloalkylene or C₃-C₆ cycloheteroalkylene;

A is hydrogen or C₁-C₆ alkyl;

n is 0, 1, 2, 3, 4 or 5; and

* represents a point of attachment.

In an embodiment, R₁ and R₂ represent independently hydrogen, lower alkyl, or halogen. In an embodiment, R₃ and R₄ represent independently hydrogen or unsubstituted C₁-C₆ alkyl. In an embodiment, Q is

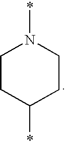

In an embodiment, R₅ is substituted aryl; R₆ is hydrogen; and A is H. In an embodiment, n is 0 or 1.

In an embodiment, R₅, R₆, and A, taken together with their bonded carbons, form an aromatic ring, e.g., phenyl, naphthyl, diphenylmethyl, biaryl; that is optionally substituted on the adjacent carbon atoms to form a bicyclic ring with a 5- or 6-membered unsaturated or saturated ring such as

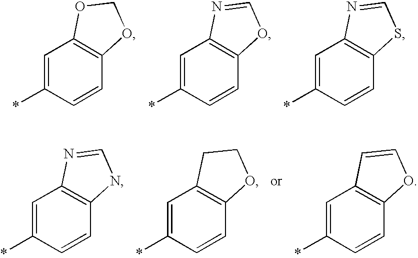

In an embodiment, R₁ is H, —CH₃, —CH(CH₃)₂, or Cl. In another embodiment, R₂ is H, Cl, lower alkyl, e.g., straight or branched C₁, C₂, C₃ (e.g., iso- or tert-butyl), C₄ or C₅ alkyl, or aryl, e.g., phenyl or fluorophenyl. R₁ and R₂ may also, taken together with the bonded carbons from the thieno, form a cyclohexyl ring. The Q group is preferably an N-substituted alkyl or cycloalkyl. The linking group denoted by ( )ₙ may be substituted or unsubstituted, straight or branched, and may be a single bond, or made up of 1, 2, 3, 4 or 5 carbons or more. In certain embodiments, n is 2, 3, 4 or 5. In certain embodiments, A is H or —CH₃. In certain embodiments, A is H.

In an embodiment, the compound has the following formula:

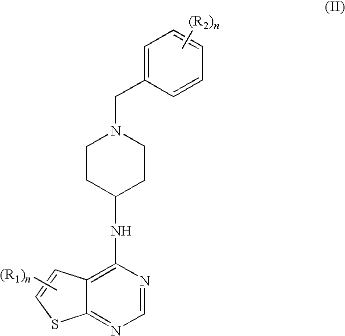

including pharmaceutically acceptable salts, solvates, and/or esters thereof; wherein R₁ represents independently for each occurrence halogen, lower alkyl, cyano, or trihalomethyl;

R₂ represents independently for each occurrence hydrogen, halogen, cyano, trihalomethyl, lower alkoxy, carboxylate, amide, or a sulfonyl group; and n represents independently for each occurrence 1 or 2.

In an embodiment, when n is 1, R₂ is not hydrogen, and when n is 2, both R₂ groups are not hydrogen. Examples of amides include amido, N-methylamido and dimethylamido groups; examples of sulfonyl groups include trifluoromethylsulfonyl, sulfonyl, and methylsulfonyl groups. In certain embodiments, the pharmaceutically acceptable salt is a maleate, hydrochloride, or fumarate salt.

In an embodiment, the compound has the following formula:

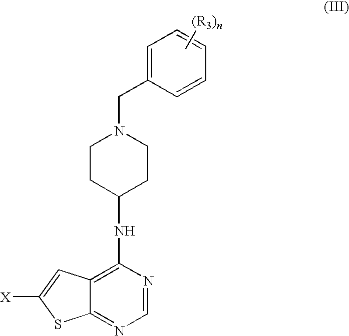

including pharmaceutically acceptable salts, solvates, and/or esters thereof; wherein, X is halogen; R₃ represents independently for each occurrence halogen, cyano, or trihalomethyl; and n is 1 or 2. In certain embodiments, the pharmaceutically acceptable salt is a maleate, hydrochloride, or fumarate salt.

In an embodiment, the compound is 5-((4-(6-chlorothieno[2,3-d]pyrimidin-4-ylamino)piperidin-1-yl)methyl)-2-fluorobenzonitrile or a pharmaceutically acceptable salt thereof.

In an embodiment, the compound is 3-((4-(6-chlorothieno[2,3-d]pyrimidin-4-ylamino)piperidin-1-yl)methyl)benzonitrile or a pharmaceutically acceptable salt thereof.

In an embodiment, the subject is a mammal. In an embodiment, the subject is a human. The compound may be administered in dosages as to be determined by one of skill in the art or as described herein. In an embodiment, the compound of formula I is administered at a dosage in the range of about 20 mg to about 1000 mg. In an embodiment, the mode of administration of the compound is oral, intravenous, sublingual, ocular, transdermal, rectal, topical, intramuscular, intra-arterial, subcutaneous, buccal, nasal, or direct delivery to the liver.

Another aspect of the invention relates to a method of treating or preventing fibrosis of an organ of a subject, comprising administering to a subject in need thereof a therapeutically effective amount of 5-((4-(6-chlorothieno[2,3-d]pyrimidin-4-ylamino)piperidin-1-yl)methyl)-2-fluorobenzonitrile, 3-((4-(6-chlorothieno[2,3-d]pyrimidin-4-ylamino)piperidin-1-yl)methyl)benzonitrile, or a pharmaceutically acceptable salt thereof. In an embodiment, the organ is the liver, kidney, or lung. In an embodiment, the subject is a human. In an embodiment, the salt is a maleate, hydrochloride, or fumarate salt.

Another aspect of the invention relates to a method of inducing apoptosis of activated hepatic stellate cells in a subject, comprising administering to a subject an effective amount of 5-((4-(6-chlorothieno[2,3-d]pyrimidin-4-ylamino)piperidin-1-yl)methyl)-2-fluorobenzonitrile, 3-((4-(6-chlorothieno[2,3-d]pyrimidin-4-ylamino)piperidin-1-yl)methyl)benzonitrile, or a pharmaceutically acceptable salt thereof. In an embodiment, the subject is a human. In an embodiment, the salt is a maleate, hydrochloride, or fumarate salt.

Another aspect of the invention relates to a method of treating or preventing fibrosis associated with hepatitis, comprising administering to a subject in need thereof a therapeutically effective amount of a compound of formula I or a composition comprising a therapeutically effective amount of a compound of formula I, wherein formula I is represented by:

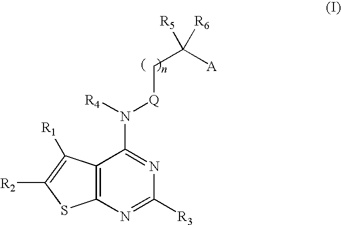

including pharmaceutically acceptable salts, solvates, and/or esters thereof; wherein

R₁ and R₂ represent independently hydrogen, lower alkyl, C₁-C₆ cycloalkyl or cycloheteroalkyl, halogen, halo-substituted alkyl, —COOH, —CN, —NH₂, —NO₂, —OH, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or

R₁ and R₂, taken together with their bonded carbon atoms, form a substituted or unsubstituted C₄-C₇ cycloalkyl or cycloheteroalkyl; wherein the C₄-C₇ cycloheteroalkyl comprises at least one of O, N or S, and the substituted C₄-C₇ cycloalkyl or cycloheteroalkyl comprises at least one substituent selected from halogen, —COOH, —CN, —NH₂, —NO₂, —OH, lower alkyl, substituted lower alkyl, substituted or unsubstituted C₁-C₆ cycloalkyl or cycloheteroalkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, and —R₉-haloalkoxy;

R₃ is H, halogen, —CN, —NH₂, lower alkyl, R₇, —OR₇, —NHR₇, —N(R₇)₂, or substituted or unsubstituted aryl or heteroaryl;

R₄ is H, R₇, or substituted or unsubstituted aryl or heteroaryl;

Q is

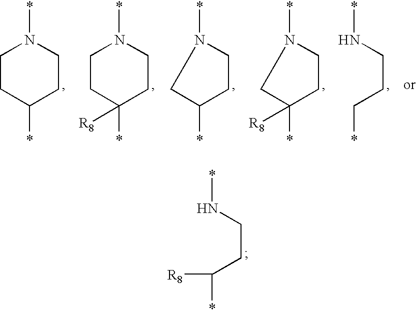

R₅ and R₆ represent independently hydrogen, halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or

R₅, R₆, and A taken together with their bonded carbons, form a substituted or unsubstituted unsaturated 5- or 6-membered carbocyclic ring or a substituted or unsubstituted saturated 5-, 6-, or 7-membered carbocyclic ring, wherein the carbocyclic ring may be a fused biaryl ring or a heterocarbocyclic ring comprising at least one heteroatom selected from the group consisting of O, N, S and P; and the substituted ring comprises at least one of halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or R₅, R₆, and A, taken together with their bonded carbons, form an aromatic ring that is optionally substituted on the adjacent carbon atoms to form a bicyclic ring with a 5- or 6-membered unsaturated or saturated ring;

R₇ represents independently for each occurrence substituted or unsubstituted C₁-C₆ alkyl or C₃-C₆ cycloalkyl or C₃-C₆ cycloheteroalkyl;

R₈ is hydrogen, halogen, CN, or a substituted or unsubstituted lower alkyl;

R₉ represents independently for each occurrence substituted or unsubstituted C₁-C₆ alkylene or C₃-C₆ cycloalkylene or C₃-C₆ cycloheteroalkylene;

A is hydrogen or C₁-C₆ alkyl;

n is 0, 1, 2, 3, 4 or 5; and

* represents a point of attachment.

In an embodiment, R₁ and R₂ represent independently hydrogen, lower alkyl, or halogen. In an embodiment, R₃ and R₄ represent independently hydrogen or unsubstituted C₁-C₆ alkyl. In an embodiment, Q is

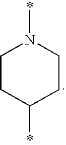

In an embodiment, R₅ is substituted aryl; R₆ is hydrogen; and A is H. In an embodiment, n is 0 or 1.

In an embodiment, the compound has the following formula:

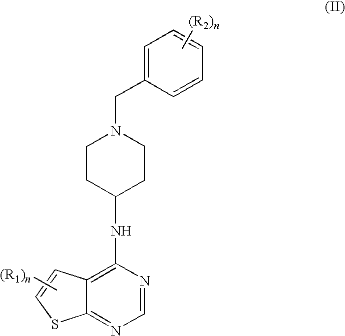

including pharmaceutically acceptable salts, solvates, and/or esters thereof; wherein R₁ represents independently for each occurrence halogen, lower alkyl, cyano, or trihalomethyl; R₂ represents independently for each occurrence hydrogen, halogen, cyano, trihalomethyl, lower alkoxy, carboxylate, amide, or a sulfonyl group; and n represents independently for each occurrence 1 or 2.

In an embodiment, the compound is 5-((4-(6-chlorothieno[2,3-d]pyrimidin-4-ylamino)piperidin-1-yl)methyl)-2-fluorobenzonitrile or a pharmaceutically acceptable salt thereof. In an embodiment, the compound is 3-((4-(6-chlorothieno[2,3-d]pyrimidin-4-ylamino)piperidin-1-yl)methyl)benzonitrile or a pharmaceutically acceptable salt thereof. In an embodiment, the organ is the liver. In an embodiment, the hepatitis is hepatitis C.

### BRIEF DESCRIPTION OF THE FIGURES

FIG. 1 depicts the results of an assay measuring apoptosis in activated rat activated hepatic stellate cells upon administration of spiperone (SP) or compound A (EP).

FIG. 2 depicts the results of an assay measuring apoptosis in activated rat activated hepatic stellate cells upon administration of spiperone (SP) or compound A (EP).

FIG. 3 depicts the results of an assay measuring apoptosis in activated rat activated hepatic stellate cells upon administration of spiperone (SP) or compound A (EP).

FIG. 4 depicts the results of an assay measuring apoptosis in activated rat activated hepatic stellate cells upon administration of spiperone (SP) or compound A (EP).

FIG. 5 depicts the results of an assay measuring apoptosis in activated human stellate cells upon administration of spiperone (SP) or compound A (EP).

FIG. 6 depicts the results of an assay measuring apoptosis in activated human stellate cells upon administration of spiperone (SP) or compound A (EP).

FIG. 7 depicts the results of an assay measuring Caspase 3/7 activity in rat activated hepatic stellate cells upon administration of compound A, compared to untreated cells or cells treated with vehicle.

FIG. 8 depicts the results of a study measuring the activity of compound A against liver lesions induced by monocrotaline (MCT).

FIG. 9 depicts the results of a study measuring the activity of compound A against lung lesions induced by monocrotaline (MCT).

FIG. 10 depicts the results of a study measuring the activity of compound A against liver fibrosis that was induced using monocrotaline (MCT).

FIG. 11 depicts the results of a study measuring the activity of compound A on liver and lung pathology, including lesions and necrosis induced using monocrotaline. The top panels are samples of lung tissue, while the bottom panels are samples of liver tissue.

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

### Definitions

For convenience, certain terms used in the specification, examples, and appended claims are collected here.

“5-HT receptor modulator” or “5-HT modulator” includes compounds having effect at the 5-HT₁, 5-HT₂, 5-HT₃, 5-HT₄, 5-HT₅, 5-HT₆ or 5-HT₇ receptors, including the subtypes of each receptor type, such as 5-HT_{1A, B, C, D, E or F}; 5-HT_{2A, B or C}; and 5-HT_{5A or B}. 5-HT modulators may be agonists, partial agonists or antagonists.

“Treating”, includes any effect, e.g., lessening, reducing, modulating, or eliminating, that results in the improvement of the condition, disease, disorder, etc.

“Alkyl” includes saturated aliphatic groups, including straight-chain alkyl groups (e.g., methyl, ethyl, propyl, butyl, pentyl, hexyl, heptyl, octyl, nonyl, decyl), branched-chain alkyl groups (e.g., isopropyl, tert-butyl, isobutyl, isoamyl), cycloalkyl (e.g., alicyclic) groups (e.g., cyclopropyl, cyclopentyl, cyclohexyl, cycloheptyl, cyclooctyl), alkyl substituted cycloalkyl groups, and cycloalkyl substituted alkyl groups. “Alkyl” further includes alkyl groups which have oxygen, nitrogen, sulfur or phosphorous atoms replacing one or more hydrocarbon backbone carbon atoms. In certain embodiments, a straight chain or branched chain alkyl has six or fewer carbon atoms in its backbone (e.g., C₁-C₆ for straight chain, C₃-C₆ for branched chain), and more preferably four or fewer. Likewise, preferred cycloalkyls have from three to eight carbon atoms in their ring structure, and more preferably have five or six carbons in the ring structure. “C₁-C₆” includes alkyl groups containing one to six carbon atoms.

The term “alkyl” also includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkoxyl, alkoxycarbonyl, alkoxycarbonyloxy, alkyl, alkynyl, alkylcarbonyl, alkylcarbonyloxy, aminocarbonyl, alkylaminocarbonyl, dialkylaminocarbonyl, alkylsulfinyl, alkylthio, alkylthiocarbonyl, thiocarboxylate, arylthio, arylcarbonyl, arylcarbonyloxy, aryloxycarbonyloxy, amino (including alkylamino, dialkylamino, arylamino, diarylamino, and alkylarylamino), acylamino (including alkylcarbonylamino, arylcarbonylamino, carbamoyl and ureido), amidino, imino, azido, carboxylate, cyano, halogen, haloalkyl, haloalkoxy, cycloalkoxyl, acetamide, alkylacetamide, cycloalkylacetamide, amine, cycloamine, heterocyclyl, hydroxyl, nitro, phosphate, phosphonato, phosphinato, sulfates, sulfonato, sulfamoyl, sulfhydryl, sulfonamido, trifluoromethyl, alkylaryl, or an aromatic or heteroaromatic moiety, or any other substituent or its equivalent disclosed herein. Cycloalkyls can be further substituted, e.g., with the substituents described herein and or their equivalents known in the art. An “alkylaryl” or an “aralkyl” moiety is an alkyl substituted with an aryl (e.g., phenylmethyl (benzyl)). “Alkyl” also includes the side chains of natural and unnatural amino acids.

A “substituted” moiety is non-limiting as to the type of substituent. As used herein, a substituent includes any one or more chemical moieties disclosed herein, or any equivalent known in the art.

“Aryl” includes groups with aromaticity, including 5- and 6-membered “unconjugated”, or single-ring, aromatic groups that may include from zero to four heteroatoms, as well as “conjugated”, or multicyclic, systems with at least one aromatic ring. Examples of aryl groups include benzene, phenyl, benzoxazole, benzthiazole, benzo[d][1,3]dioxole, naphthyl, quinolinyl, pyrrole, furan, thiophene, thiazole, isothiazole, imidazole, triazole, tetrazole, pyrazole, oxazole, isooxazole, pyridine, pyridinyl, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety, or any other substituent disclosed herein or its equivalent. Aryl groups can also be fused or bridged with alicyclic or heterocyclic rings which are not aromatic so as to form a multicyclic system (e.g., tetralin, methylenedioxyphenyl).

“Alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl), branched-chain alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “alkenyl” further includes alkenyl groups which include oxygen, nitrogen, sulfur or phosphorous atoms replacing one or more hydrocarbon backbone carbons. In certain embodiments, a straight chain or branched chain alkenyl group has six or fewer carbon atoms in its backbone (e.g., C₂-C₆ for straight chain, C₃-C₆ for branched chain.) Likewise, cycloalkenyl groups may have from three to eight carbon atoms in their ring structure, and more preferably have five or six carbons in the ring structure. The term “C₂-C₆” includes alkenyl groups containing two to six carbon atoms.

The term “alkenyl” also includes both “unsubstituted alkenyls” and “substituted alkenyls”, the latter of which refers to alkenyl moieties having substituents replacing a hydrogen on one or more hydrocarbon backbone carbon atoms. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight-chain alkynyl groups (e.g., ethynyl, propynyl, butynyl, pentynyl, hexynyl, heptynyl, octynyl, nonynyl, decynyl), branched-chain alkynyl groups, and cycloalkyl or cycloalkenyl substituted alkynyl groups. The term “alkynyl” further includes alkynyl groups having oxygen, nitrogen, sulfur or phosphorous atoms replacing one or more hydrocarbon backbone carbons. In certain embodiments, a straight chain or branched chain alkynyl group has six or fewer carbon atoms in its backbone (e.g., C₂-C₆ for straight chain, C₃-C₆ for branched chain). The term “C₂-C₆” includes alkynyl groups containing two to six carbon atoms.

The term “alkynyl” also includes both “unsubstituted alkynyls” and “substituted alkynyls”, the latter of which refers to alkynyl moieties having substituents replacing a hydrogen on one or more hydrocarbon backbone carbon atoms. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” includes an alkyl group, as defined above, but having from one to ten, more preferably from one to six, carbon atoms in its backbone structure. “Lower alkenyl” and “lower alkynyl” have chain lengths of, for example, 2-5 carbon atoms.

“Acyl” includes compounds and moieties which contain the acyl radical (CH₃CO—) or a carbonyl group. “Substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Acylamino” includes moieties wherein an acyl moiety is bonded to an amino group. For example, the term includes alkylcarbonylamino, arylcarbonylamino, carbamoyl and ureido groups.

“Aroyl” includes compounds and moieties with an aryl or heteroaromatic moiety bound to a carbonyl group. Examples of aroyl groups include phenylcarboxy, naphthyl carboxy, etc.

“Alkoxyalkyl”, “alkylaminoalkyl” and “thioalkoxyalkyl” include alkyl groups, as described above, which further include oxygen, nitrogen or sulfur atoms replacing one or more hydrocarbon backbone carbon atoms, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, and trichloromethoxy.

The terms “heterocyclyl” or “heterocyclic group” include closed ring structures, e.g., 3- to 10-, or 4- to 7-membered rings, which include one or more heteroatoms. Heterocyclyl groups can be saturated or unsaturated and include pyrrolidine, oxolane, thiolane, piperidine, piperizine, morpholine, lactones, lactams such as azetidinones and pyrrolidinones, sultams, sultones, and the like. The heterocyclic ring can be substituted at one or more positions with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or heteroatoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

“Polycyclyl” or “polycyclic radical” refers to two or more cyclic rings (e.g., cycloalkyls, cycloalkenyls, cycloalkynyls, aryls and/or heterocyclyls) in which two or more carbons are common to two adjoining rings. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminocarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

“Heteroatom” includes atoms of any element other than carbon or hydrogen. Examples of heteroatoms include nitrogen, oxygen, sulfur and phosphorus.

“Activated hepatic stellate cell” refers to a hepatic stellate cell that has undergone a transformation in response to injury. Activated hepatic stellate cells are typically myofibroblast-like, express smooth muscle alpha-actin, have enhanced collagen production, and/or express tissue inhibitor of metalloproteinases-1. Generally, these activated cells are relatively resistant to apoptosis and undergo proliferation, producing a growing population of profibrogenic cells.

It will be noted that the structure of some of the compounds of the invention includes asymmetric carbon atoms. It is to be understood accordingly that the isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Furthermore, the structures and other compounds and moieties discussed in this application also include all tautomers thereof. Alkenes can include either the E- or Z-geometry, where appropriate.

“Combination therapy” (or “co-therapy”) includes the administration of a 5-HT modulator of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In an embodiment, the combination therapy comprises a piperidinylamino-thieno[2,3-d]pyrimidine compound described herein and an anti-inflammatory agent. In an embodiment, the anti-inflammatory agent is aspirin, salsalate, diflunisal, ibuprofen, ketoprofen, nabumetone, piroxicam, naproxen, diclofenac, indomethacin, sulindac, tolmetin, etodolac, ketorolac, oxaprozin, or celecoxib.

In an embodiment, the combination therapy comprises a piperidinylamino-thieno[2,3-d]pyrimidine compound described herein and a protease inhibitor. In an embodiment, the protease inhibitor is teleprevir or BILLN 2061. In an embodiment, the combination therapy further comprises ribavirin. In an embodiment, the combination therapy further comprises an interferon. In an embodiment, the interferon is a pegylated interferon.

In an embodiment, the combination therapy comprises a piperidinylamino-thieno[2,3-d]pyrimidine compound described herein and a polymerase inhibitor. In an embodiment, the polymerase inhibitor is NM 283.

In an embodiment, the combination therapy comprises a piperidinylamino-thieno[2,3-d]pyrimidine compound described herein and an interferon. In an embodiment, the interferon is interferon-α. In an embodiment, the interferon is pegylated. In an embodiment, the interferon is pegylated interferon-α-2b sold under the tradename PEGINTRON™. In an embodiment, the interferon is pegylated interferon-α-2a sold under the tradename PEGASYS®. In an embodiment, the combination therapy further comprises ribavirin, the chemical compound having the name 1-(β-D-Ribofuranosyl)-1H-1,2,4-triazole-3-carboxamide.

In an embodiment, the combination therapy comprises a piperidinylamino-thieno[2,3-d]pyrimidine compound described herein and ribavirin.

In an embodiment, the combination therapy comprises a piperidinylamino-thieno[2,3-d]pyrimidine compound described herein and a second agent selected from the group consisting of helicase inhibitor, ribozyme, antisense therapy, and T-cell-based therapeutic.

In an embodiment, the combination therapy is used to treat hepatitis, such as hepatitis C.

An “anionic group,” as used herein, refers to a group that is negatively charged at physiological pH. Preferred anionic groups include carboxylate, sulfate, sulfonate, sulfinate, sulfamate, tetrazolyl, phosphate, phosphonate, phosphinate, or phosphorothioate or functional equivalents thereof. “Functional equivalents” of anionic groups are intended to include bioisosteres, e.g., bioisosteres of a carboxylate group. Bioisosteres encompass both classical bioisosteric equivalents and non-classical bioisosteric equivalents. Classical and non-classical bioisosteres are known in the art (see, e.g., Silverman, R. B. The Organic Chemistry of Drug Design and Drug Action, Academic Press, Inc.: San Diego, Calif., 1992, pp. 19-23). A particularly preferred anionic group is a carboxylate.

The term “heterocyclic group” is intended to include closed ring structures in which one or more of the atoms in the ring is an element other than carbon, for example, nitrogen, or oxygen or sulfur. Heterocyclic groups can be saturated or unsaturated and heterocyclic groups such as pyrrole and furan can have aromatic character. They include fused ring structures such as quinoline and isoquinoline. Other examples of heterocyclic groups include pyridine and purine. Heterocyclic groups can also be substituted at one or more constituent atoms with, for example, a halogen, a lower alkyl, a lower alkenyl, a lower alkoxy, a lower alkylthio, a lower alkylamino, a lower alkylcarboxyl, a nitro, a hydroxyl, —CF₃, —CN, or the like.

The compounds described herein, such as those of formula III, may be highly selective. For example, 5-((4-(6-Chlorothieno[2,3-d]pyrimidin-4-ylamino)piperidin-1-yl)methyl)-2-fluorobenzonitrile (“compound A”) is a highly selective and potent (Kᵢ=1.8 nM) 5-HT_{2B} receptor antagonist with more than 500-fold differences in receptor affinities for compared with all other 5-HT receptor subtypes, except for the 5-HT_{1A} (Kᵢ=100 nM) receptor. This compound has almost no affinity (Kᵢ>1 μM) for more than 51 receptors tested including GPCRs, ion channels and receptor tyrosine kinases; and is active on the dopamine D4.4 receptor (Ki=5.4 nM) and displays moderate activity for the dopamine D3 receptor (Kᵢ˜310 nM). However, blocking of the dopamine D3 and D4 receptors is not associated with extrapyramidal side effects. Compound A appears to be a weak dopamine D2 receptor (IC₅₀=0.67 μM) antagonist and did not show any dopamine D1 and D5 receptor activity (Kᵢ>5 μM). Compound A displayed moderate binding to the σ1 and σ2 receptors (Ki=100 nM and 110 nM, respectively). However, in functional assays the compound demonstrated very weak agonist activity for σ receptors (EC50≈10 μM.

As described above, fibrosis affects numerous bodily organs, including the liver, kidneys, lungs, and heart. Fibrosis of these organs can be caused by or associated with a variety of disorders or conditions. For example, liver fibrosis can be caused by parasitic infection, trauma, autoimmune diseases, alcoholism, viral infection, hypoxia, sepsis, bacterial infection, non-alcoholic fatty liver disease, non-alcoholic steatohepatitis, and as a side effect of taking certain medications, e.g., acetaminophen. Accordingly, one aspect of the invention relates to a method of treating or preventing liver fibrosis associated with such diseases or conditions by administering a therapeutically effective amount of a compound described herein to a patient in need of treatment. In particular, one aspect of the invention relates to a method of treating or preventing liver fibrosis associated with hepatitis A, B, or C. In another aspect, the invention relates to a method of treating or preventing liver injury associated with necrosis, inflammation, or abnormal apoptosis, comprising administering a therapeutically effective amount of a compound described herein to a subject in need of treatment.

Kidney fibrosis is associated with a variety of disorders including acute kidney disease, chronic kidney disease, renal failure, hypertension, and as a side effect of taking certain medications. Accordingly, one aspect of the invention relates to a method of treating or preventing kidney fibrosis associated with such disorders by administering a therapeutically effective amount of a compound described herein to a patient in need of treatment. Kidney fibrosis is also associated with renal transplant in some patients. Accordingly, one aspect of the invention relates to a method of treating or preventing kidney fibrosis associated with renal transplant by administering a therapeutically effective amount of a compound described herein to a patient in need of treatment.

Lung fibrosis is characterized by the abnormal accumulation of fibrous tissue in the lung. Lung fibrosis can be caused by or associated with a variety of disorders or conditions. For example, lung fibrosis has been associated with certain autoimmune disorders, smoking, exposure to certain airborne pollutants, taking certain medications, and exposure to certain forms of therapeutic radiation. Accordingly, one aspect of the invention relates to a method of treating or preventing lung fibrosis associated with such disorders or conditions by administering a therapeutically effective amount of a compound described herein to a patient in need of treatment. In an embodiment, the lung fibrosis is associated with smoking, e.g., smoking tobacco.

Heart fibrosis can be associated with hypertension. Accordingly, one aspect of the invention relates to a method of treating or preventing heart fibrosis associated with hypertension by administering a therapeutically effective amount of a compound described herein to a patient in need of treatment. In an embodiment, the heart fibrosis is myocardial fibrosis or endocardial fibrosis. In an embodiment, one aspect of the invention relates to a method of treating or preventing hypertension by administering a therapeutically effective amount of a compound described herein to a patient in need of treatment. In an embodiment, the hypertension is portal hypertension or pulmonary hypertension.

The compounds of the invention may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For example, the compound may be administered at a dosage in the range of about 20 mg to about 1000 mg.

It will be appreciated that the amount of the compound of the invention required for use in any treatment will vary not only with the particular compounds or composition selected but also with the route of administration, the nature of the condition being treated, and the age and condition of the patient, and will ultimately be at the discretion of the attendant physician.

The compositions and combination therapies of the invention may be administered in combination with a variety of pharmaceutical excipients, including stabilizing agents, carriers and/or encapsulation formulations as described herein.

Aqueous compositions of the present invention comprise an effective amount of the peptides of the invention, dissolved or dispersed in a pharmaceutically acceptable carrier or aqueous medium.

“Pharmaceutically or pharmacologically acceptable” include molecular entities and compositions that do not produce an adverse, allergic or other untoward reaction when administered to an animal, or a human, as appropriate. “Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For human administration, preparations should meet sterility, pyrogenicity, general safety and purity standards as required by FDA Office of Biologics standards.

The compositions and combination therapies of the invention may be formulated for parenteral administration, e.g., formulated for injection via the intravenous, intramuscular, subcutaneous, intralesional, or even intraperitoneal routes. The preparation of an aqueous composition that contains a composition of the invention or an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Therapeutic or pharmacological compositions of the present invention will generally comprise an effective amount of the component(s) of the combination therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including cremes, lotions, mouthwashes, inhalants and the like.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for intramuscular injection is also contemplated. In this regard, the use of DMSO as solvent is preferred as this will result in extremely rapid penetration, delivering high concentrations of the active compound(s) or agent(s) to a small area.

The use of sterile formulations, such as saline-based washes, by surgeons, physicians or health care workers to cleanse a particular area in the operating field may also be particularly useful. Therapeutic formulations in accordance with the present invention may also be reconstituted in the form of mouthwashes, or in conjunction with antifungal reagents. Inhalant forms are also envisioned. The therapeutic formulations of the invention may also be prepared in forms suitable for topical administration, such as in cremes and lotions.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals. For example, for parenteral administration, a suitably buffered, and if necessary, isotonic aqueous solution would be prepared and used for intravenous, intramuscular, subcutaneous or even intraperitoneal administration. One dosage could be dissolved in 1 ml of isotonic NaCl solution and either added to 1000 ml of hypodermolysis fluid or injected at the proposed site of infusion, (see for example, Remington's Pharmaceutical Sciences 15th Edition, pages 1035-1038 and 1570-1580).

In certain embodiments, active compounds may be administered orally. This is contemplated for agents which are generally resistant, or have been rendered resistant, to proteolysis by digestive enzymes. Such compounds are contemplated to include chemically designed or modified agents; dextrorotatory peptides; and peptide and liposomal formulations in time release capsules to avoid peptidase and lipase degradation.

Pharmaceutically acceptable salts include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze drying techniques which yield a powder of the active ingredient plus any additional desired ingredient from a previously sterile-filtered solution thereof.

The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include, e.g., tablets or other solids for oral administration; liposomal formulations; time-release capsules; and any other form currently used, including cremes.

Additional formulations suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. Oral formulations of compounds of the invention, e.g., compounds of formula III, may desirably be formulated for once or twice-daily administration.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabensas preservatives, a dye and flavoring, such as cherry or orange flavor.

The pharmaceutical compositions of this invention may be used in the form of a pharmaceutical preparation, for example, in solid, semisolid or liquid form, which contains one or more of the compound of the invention, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

For preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions of the invention may be incorporated for administration orally or by injection include aqueous solution, suitably flavored syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

For treating clinical conditions and diseases noted above, the compound of this invention may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. In addition to the modes of administration listed above, a therapeutic agent described herein may be administered by a method comprising removing a portion of a subject's liver tissue, treating the liver tissue with the therapeutic agent, and implanting the liver tissue back into the patient.

Piperidinylamino-thieno[2,3-d]pyrimidine compounds have been generally described above with respect to generic formulae I, II, and III. In certain embodiments, the piperidinylamino-thieno[2,3-d]pyrimidine compound is one of the following:

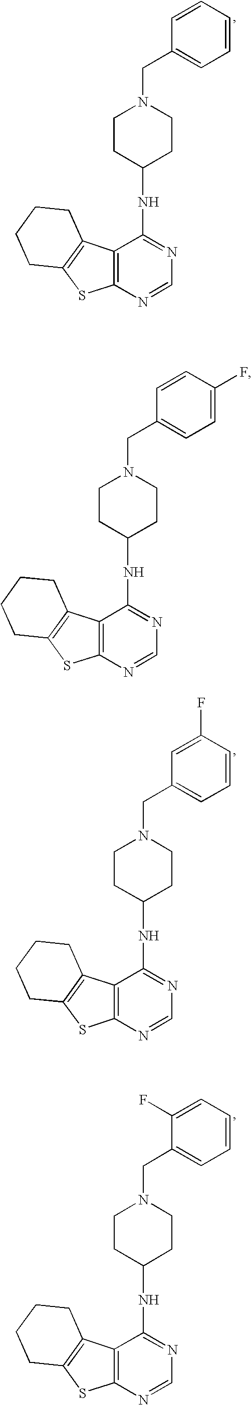

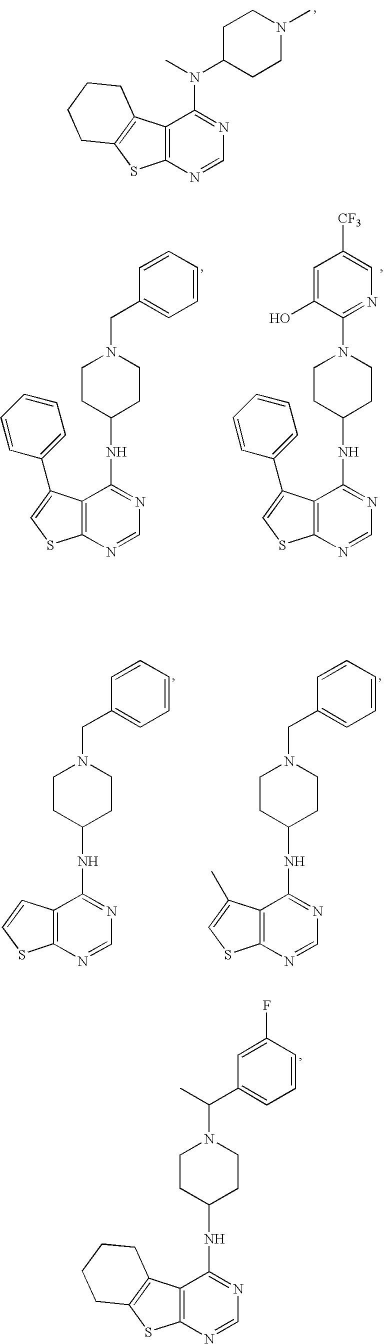

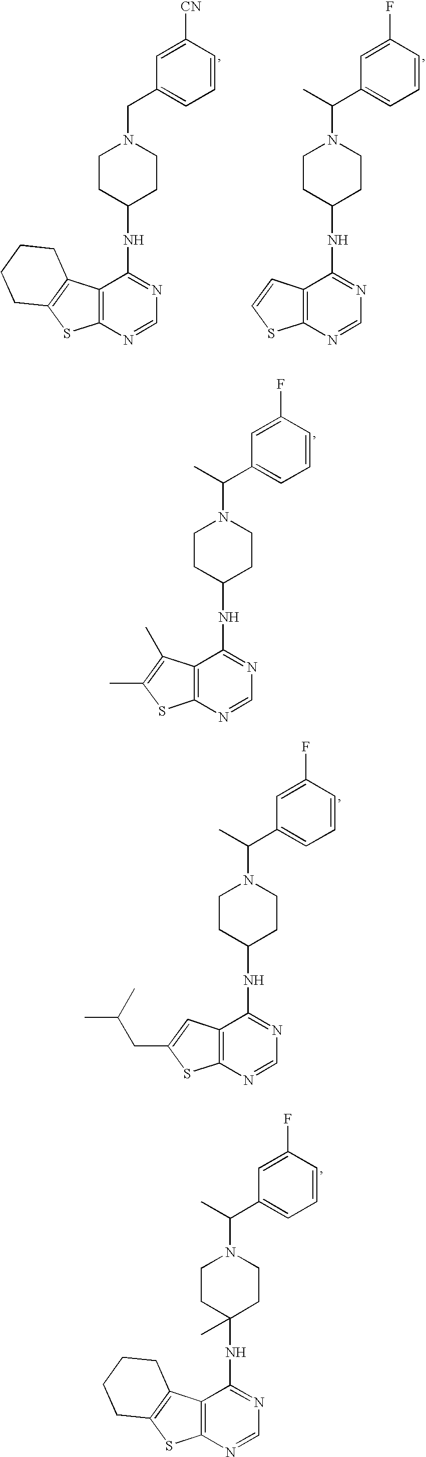

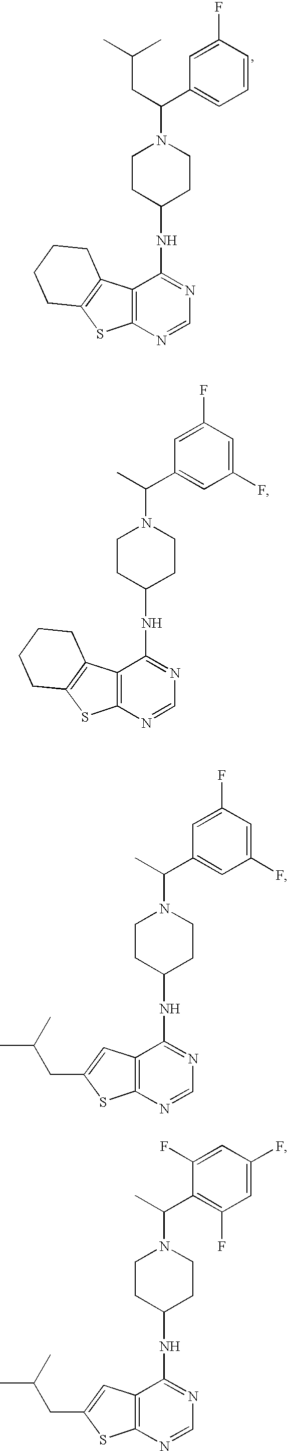

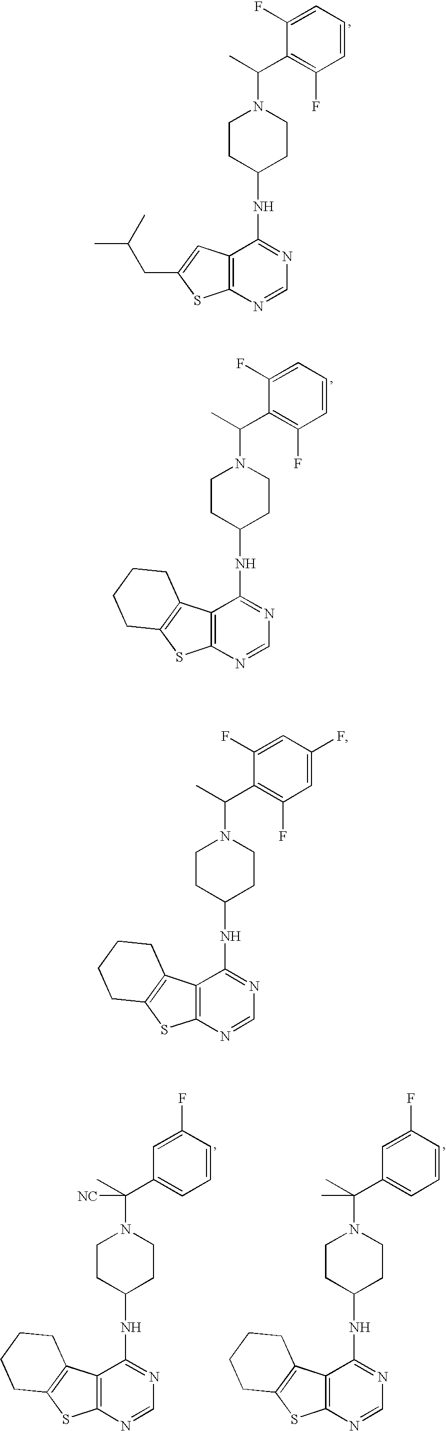

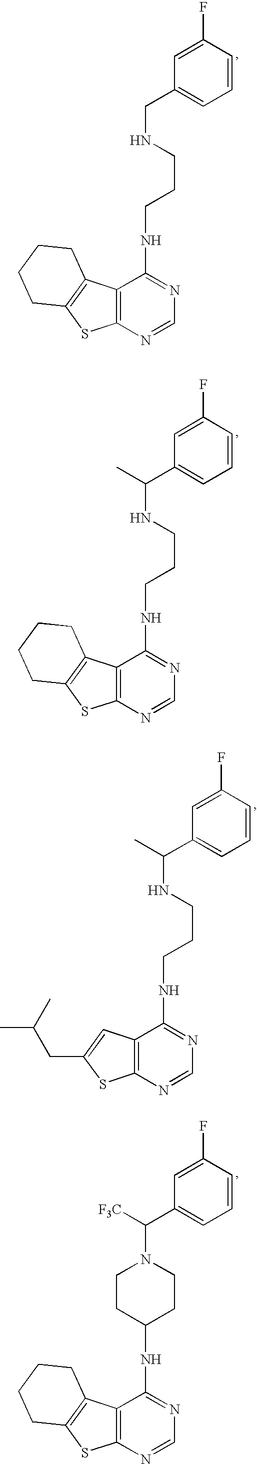

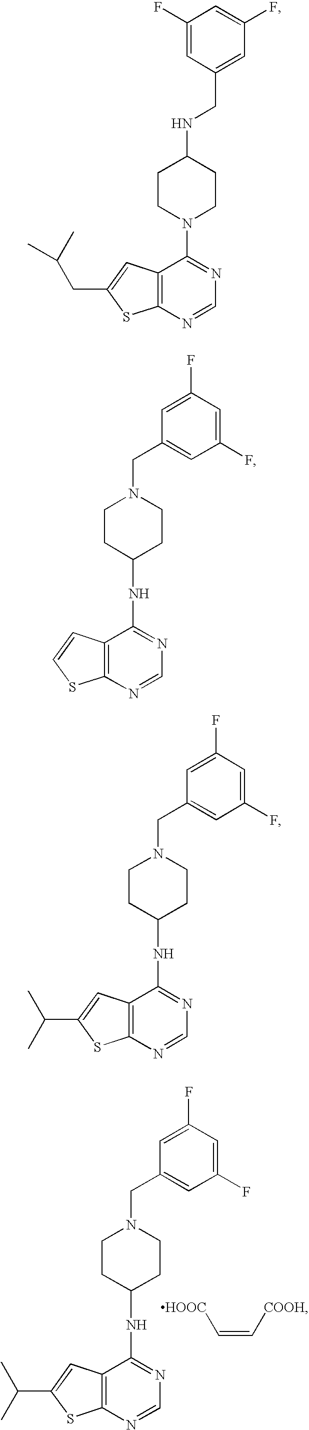

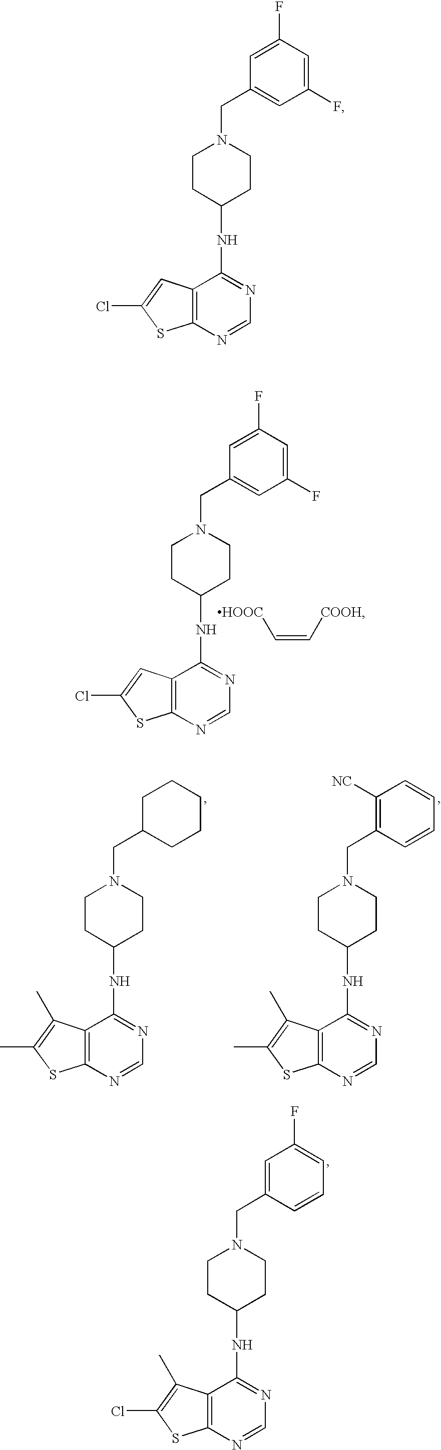

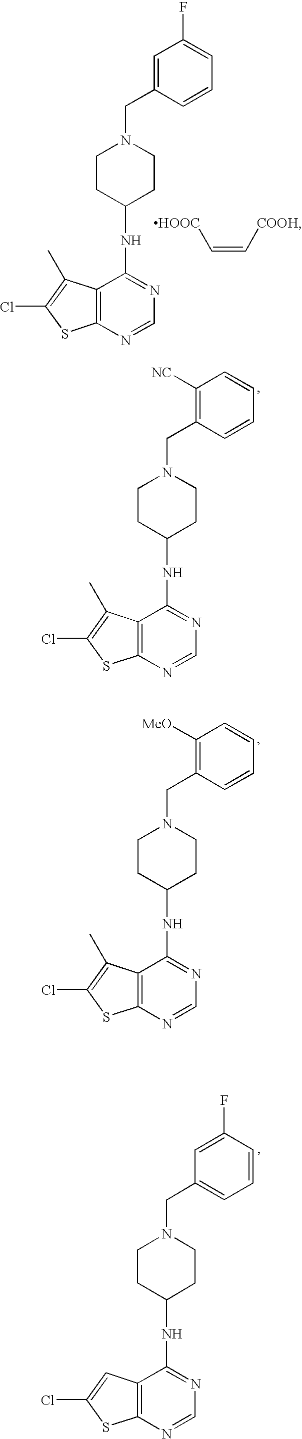

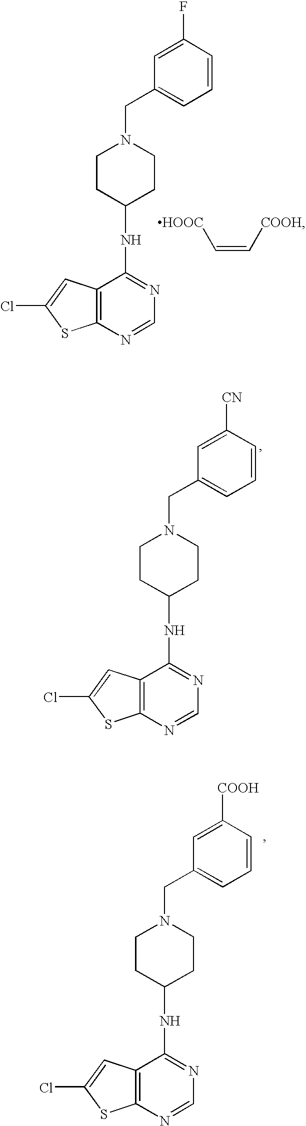

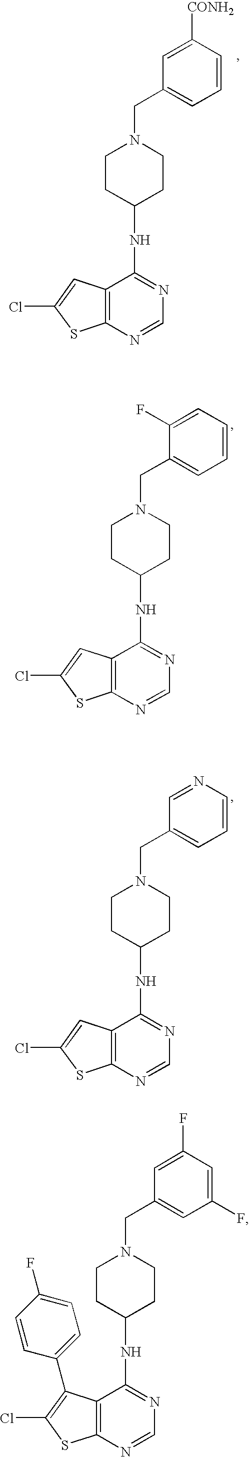

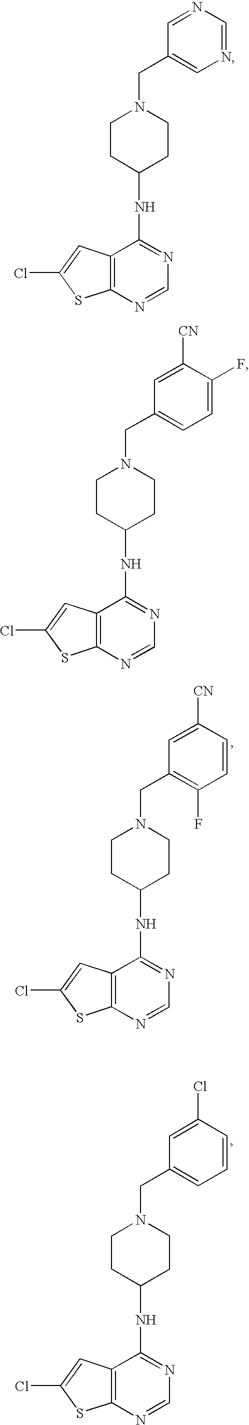

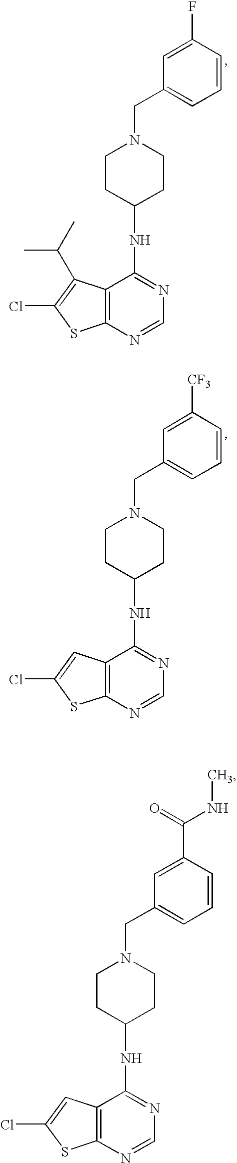

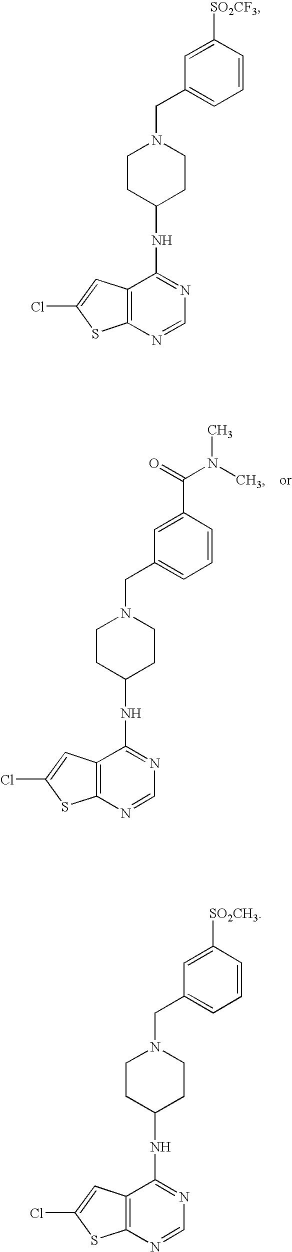

Methods for preparing piperidinylamino-thieno[2,3-d]pyrimidine compounds are illustrated below. Additional procedures can be found in US2005/0222176, which is hereby incorporated by reference.

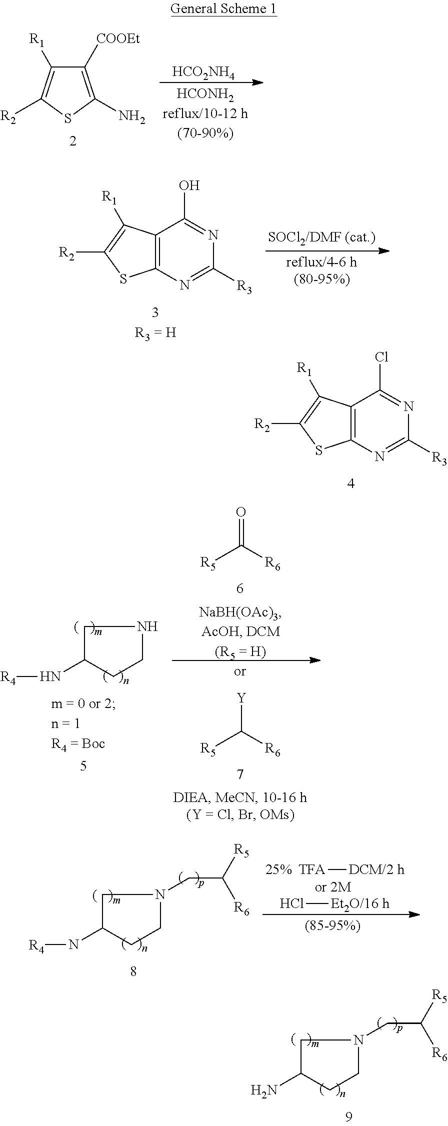

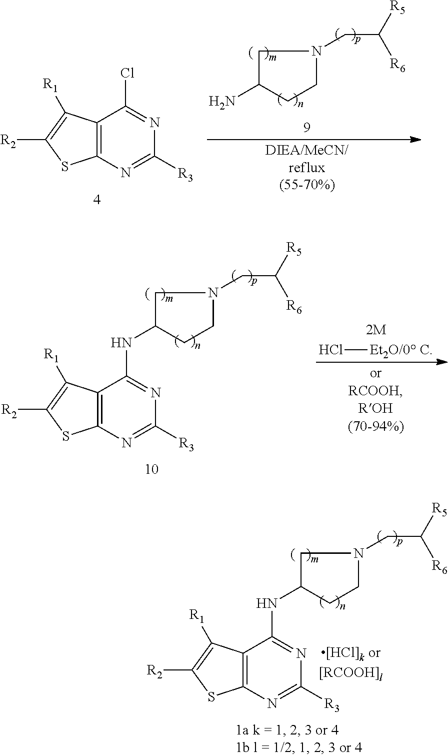

### Preparation 1:

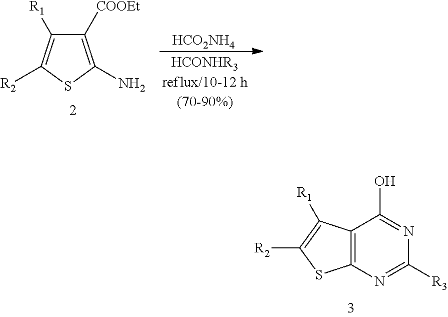

A mixture of amino ester derivative 2 (1 mmol) and ammonium formate (1.5 mmol) in formamide (4 mL) was heated at reflux for 12 h. Completion of reaction was monitored via TLC. The reaction mixture was allowed to cool to room temperature and then poured into ice (50 g) to afford a creamy precipitate. The precipitate was collected by filtration, and recrystallized from acetone/water to give 3 in 70-90% yields.

### Preparation 2:

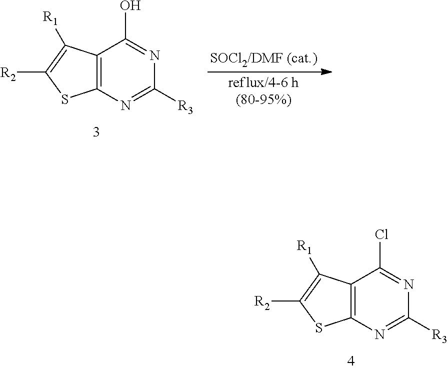

A mixture of thieno[2,3-d]pyrimidin-4-ol derivative 3 (3.7 mmol), thionyl chloride (5.5 mL) and dry DMF (0.5 mL) was heated at reflux for 4 h. The reaction mixture was cooled to room temperature and the excess thionyl chloride was removed by vacuum distillation. To the resulting residue 200 g of ice was added and extracted with dichloromethane (3×100 mL). The combined organic layers were dried (Na₂SO₄) and concentrated. The product was purified by silica chromatography (100% DCM) to afford 4-Chloro-thieno[2,3-d]-pyrimidine 4 in 80-95% yields.

### Preparation 3:

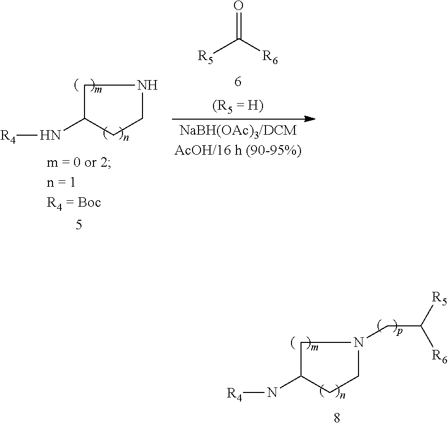

To a mixture of 4-N-Boc-amino piperidine derivative 5 (10 mmol) and aromatic aldehyde 6 (10 mmol) in 40 mL of DCM or DCE (1,2-dichloroethane) was added sodium triacetoxyborohydride (15 mmol) followed by acetic acid (20 mmol) under N₂ atmosphere. The resulting cloudy mixture was stirred at room temperature for 16 h and quenched with aq.NaHCO₃ solution. The product was extracted with EtOAc, dried (Na₂SO₄) and the solvent was evaporated to get the product 8 in 90-95% yields.

### Preparation 4:

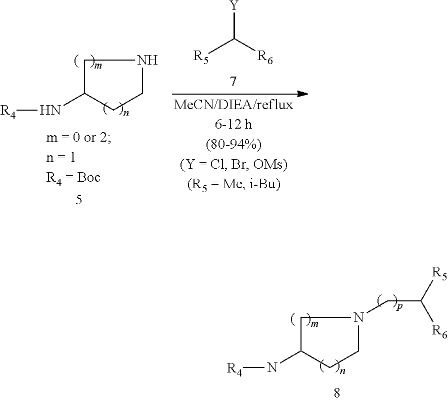

To a mixture of 4-N-Boc-amino piperidine 5 (10 mmol) and N,N-diisopropylethylamine (30 mmol) in 30 mL of CH₃CN under N₂ atmosphere was added intermediate 7 (10 mmol) at room temperature. The resulting mixture was heated at 80° C. for 16 h. The reaction mixture was quenched with aq.NaHCO₃ and the product was extracted with EtOAc. The organic extract was dried (Na₂SO₄) and the solvent was evaporated under reduced pressure to get the product 8 in 80-94% yields.

### Preparation 5:

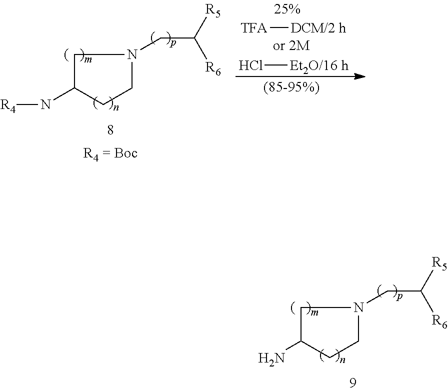

The N-Boc-protection of crude 4-N-Boc-aminobenzyl piperidine derivative 8 was removed by either treating with 25% TFA-DCM at room temperature for 2 h or with 2M HCl in Et₂O solution at room temperature for 16-20 h. In both cases, the solvent was evaporated followed by addition of dry Et₂O. The resulting precipitate was filtered, washed several times with dry Et₂O and dried under vacuum to afford the corresponding salts of 4-amino-1-benzyl piperidine derivative 9. The free base was either isolated or generated in situ during the next coupling step.

### Preparation 6:

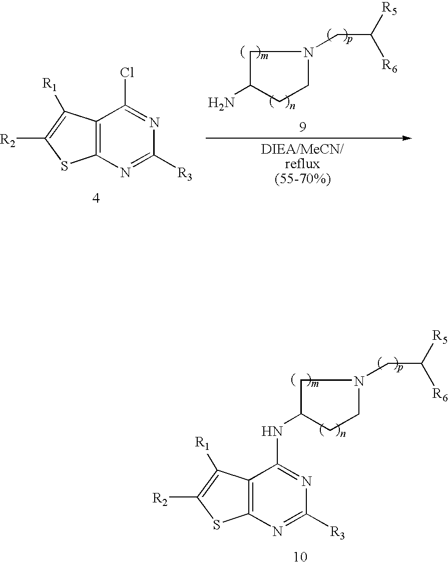

To a solution of 4-amino-piperidines 9 (1 mmol) in acetonitrile (5mL) under N₂ was added N,N-diisopropylethylamine (4 mmol) followed by chloro-thienopyrimidine 4 (1 mmol). The resulting solution was heated at reflux for 24-48 h (monitored by TLC). The solvent was evaporated and the resulting solid was dissolved in EtOAc (20 mL) and washed with aq. NaHCO₃ (10 mL) and brine solution (10 mL). The organic layer was dried (Na₂SO₄), concentrated and purified by silica chromatography (1% MeOH in DCM) to afford 10 in 55-60% yields.

### Preparation 7:

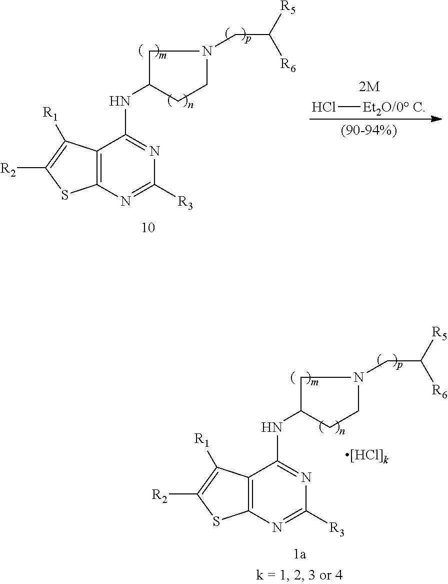

To a solution of 10 (1 mmol) in dry DCM (1 mL) was added 2 M HCl in ether (10 mL) at 0° C. and stirred at the same temperature for 1 h. The precipitated product was filtered, washed with dry Et₂O and dried under vacuum to afford pure compounds 1a in 90-94% yields.

### Preparation 8:

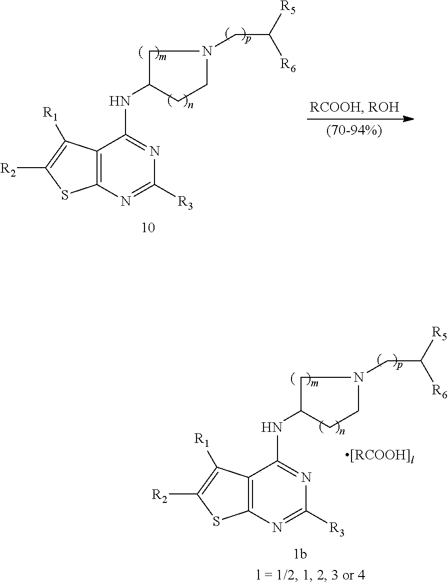

To a solution of 10 (1 mmol) in dry EtOH/DCM (2 mL) was added maleic acid (1 mmol) in EtOH (5 mL) at room temperature and stirred for 1 h. The reaction mixture was diluted with diethyl ether (5 mL) and cooled to 0° C. for 6-8 h. The precipitated product was filtered, washed with dry Et₂O and dried under vacuum to afford pure compounds 1b in 70-94% yields.

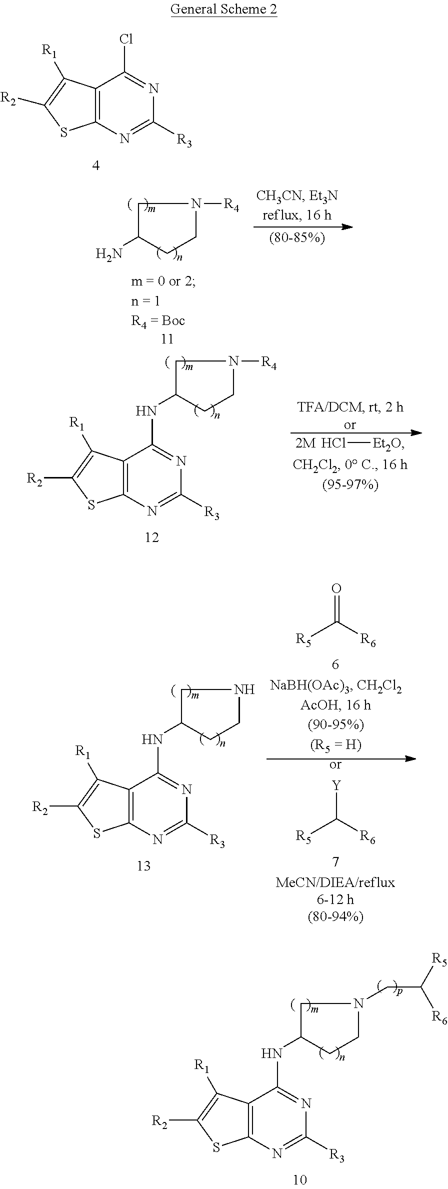

### Preparation 9:

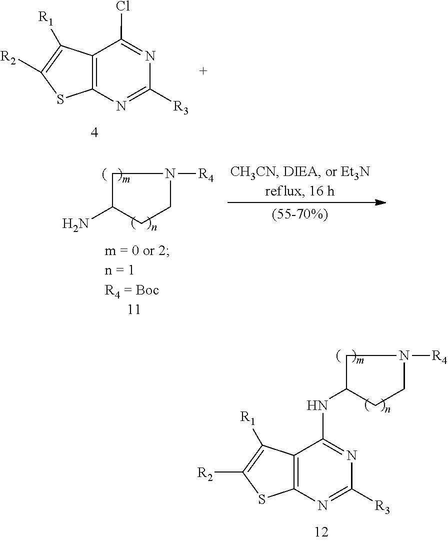

To a solution of 1-Boc-4-amino-piperidine 11 (2 mmol) in acetonitrile (5 mL) was added N,N-diisopropyl ethylamine (4 mmol) and stirred for 5 min. at room temperature under N₂. Chloro-thienopyrimidine 4 was added to the mixture and the contents were heated at reflux for 16 h (monitored by TLC). The solvent was evaporated and to the residue EtOAc (20 mL) and water (10 mL) were added. The organic layer was dried (MgSO₄) and concentrated to yield crude product. It was purified by silica chromatography (1% MeOH in DCM) to afforded the pure products 12 in 55-70% yields.

### Preparation 10:

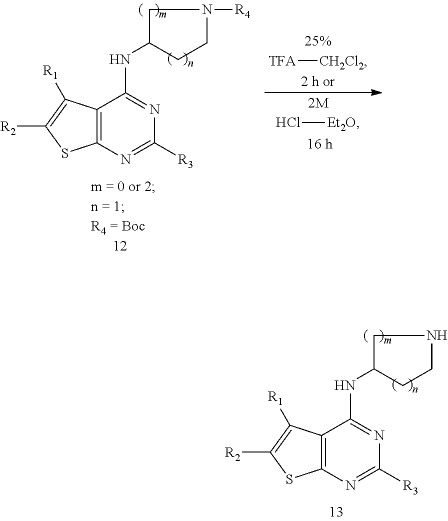

The Boc-protection of 12 was removed by either treating with 25% TFA-DCM at room temperature for 2 h or with 2 M HCl in Et₂O solution at room temperature for 16-20 h. In both cases, the solvent was evaporated followed by addition of dry Et₂O. The resulting precipitate was filtered, washed several times with dry Et₂O and dried under vacuum to afford the salts 13 in 95-97% yields. The corresponding free base was either isolated or generated in situ during the next coupling step.

### Preparation 11:

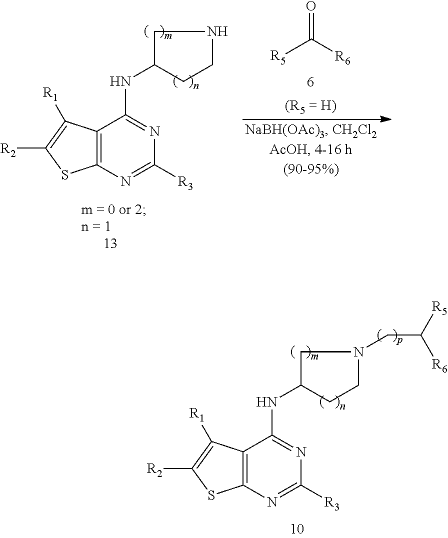

To a mixture of 13 (10 mmol) and aldehyde 6 (10 mmol) in 40 mL of DCM or DCE (1,2-dichloroethane) under N₂ atmosphere was added sodium triacetoxyborohydride (15 mmol) followed by acetic acid (20 mmol) at room temperature. The resulting cloudy mixture was stirred at room temperature for 16 h. The reaction mixture was quenched by adding aq. NaHCO₃, and the product was extracted with EtOAc. The EtOAc extract was dried (MgSO₄) and the solvent was evaporated to give the crude product. Purification by silica gel or crystallization afforded the pure products 10 in 90-95% yields.

### Preparation 12:

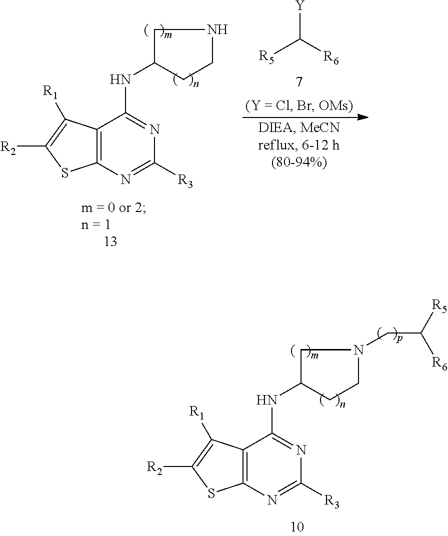

To a mixture of 13 (10 mmol) and N,N-diisopropylethylamine (30 mmol) in 30 mL of CH₃CN was added intermediate 7 (10 mmol) at room temperature under N₂ atmosphere. The resulting mixture was stirred at reflux for 16 h. The reaction mixture was quenched with aq.NaHCO₃ and the product was extracted with EtOAc. The organic extract was dried (Na₂SO₄) and the solvent was evaporated to give the product 10 in 80-94% yields.

### Example 1

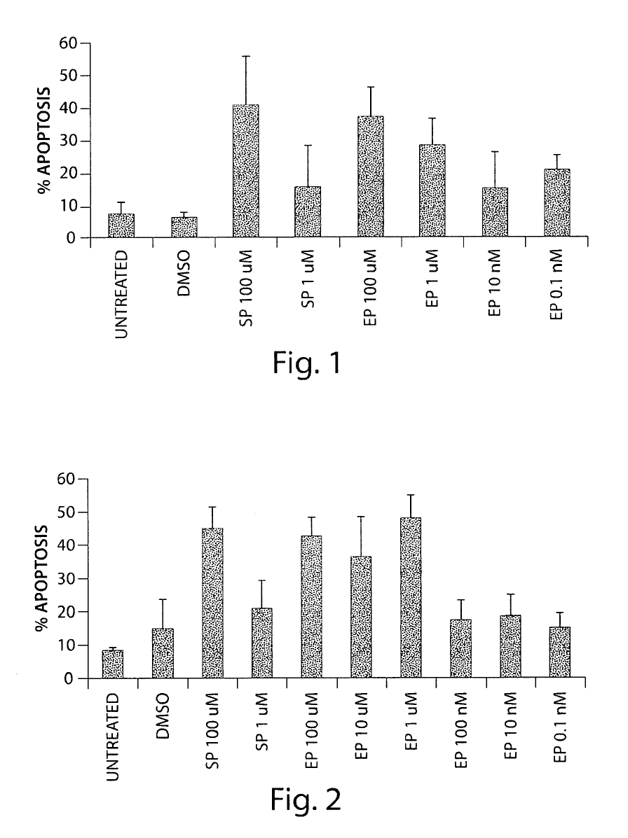

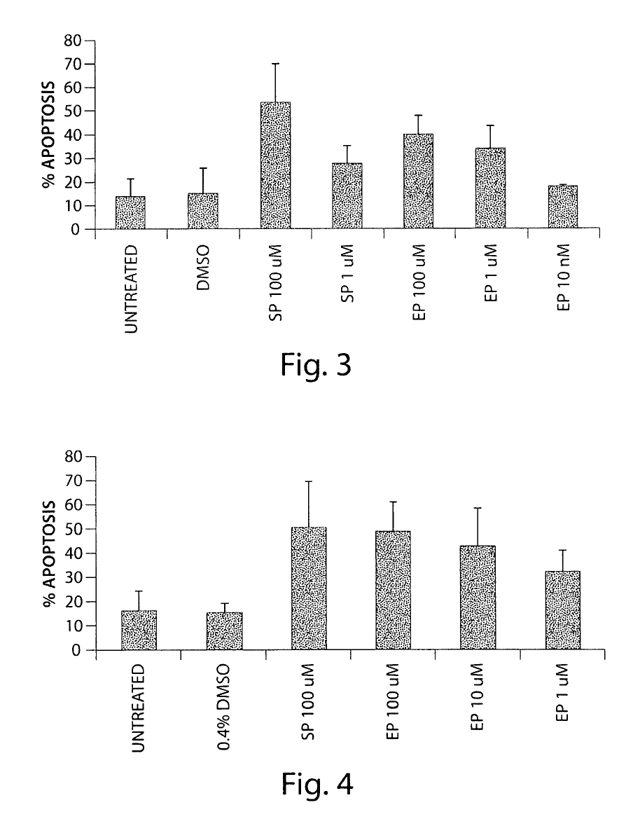

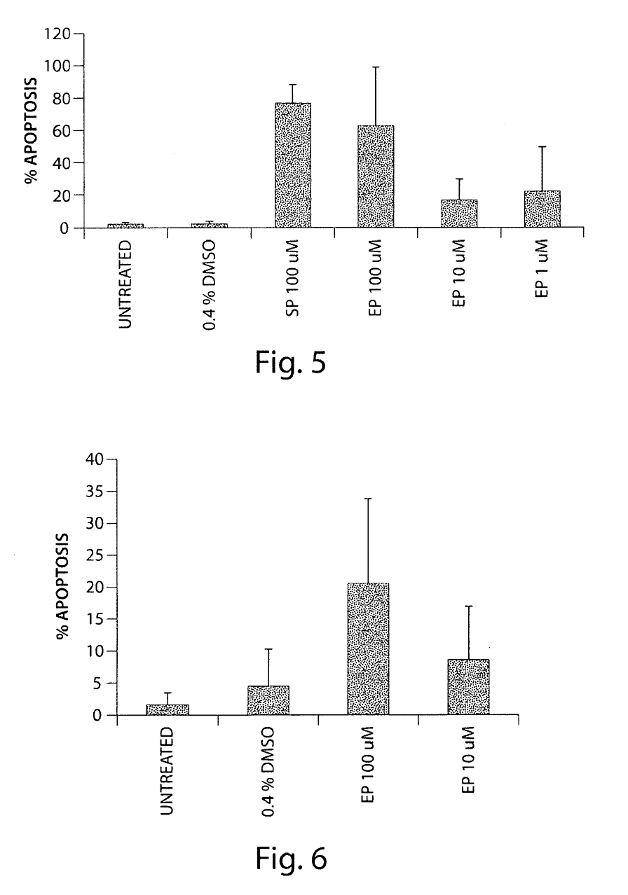

The compound 5-((4-(6-chlorothieno[2,3-d]pyrimidin-4-ylamino)piperidin-1-yl)methyl)-2-fluorobenzonitrile (hereinafter “compound A”), was evaluated for activity in promoting apoptosis of rodent and human activated hepatic myofibroblasts. The activity of compound A was compared against untreated cells, cells treated with dimethylsulfoxide, and cells treated with spiperone. Spiperone is a nonselective 5-HT_{2B} antagonist. In the acridine orange assay for morphological assessment of apoptosis, compound A dose-dependently promotes elevated rates of apoptosis of rat activated hepatic myofibroblasts (40% of cells apoptotic with 1 μM dose). The results of the acridine orange assay are depicted in FIGS. 1-4.

### Example 2

Compound A was tested on human activated hepatic myofibroblasts. The tested human cells were primary activated hepatic stellate cells isolated from normal human liver resected during removal of adjacent tumour tissue in patients with primary liver cancer. The cells were then cultured on plastic in full media and passaged at least 4 times to generate pure activated activated hepatic stellate cells, representing the major fibrogenic cell of the liver. Cultures of these cells, displayed 60% and 20% apoptosis following an overnight incubation with compound A at 100 μM and 1 μM doses, respectively.

### Example 3

Compound A was evaluated for activity in inducing Caspase 3/7 activity. Caspase 3 is involved in regulating apoptosis, and increases in Caspase 3 activity in this assay correlate to increased levels of apoptosis.

General Procedure: The assay was performed based on the following protocol:
1. Passage rHSC at an appropriate density on a 96 well format and allow to adhere overnight.
2. Treat cells with inhibitors for an appropriate length of time including the relevant controls.
3. Allow both buffer and substrate to reach room temperature.
4. Immediately prior to use, resuspend the substrate in 2.5 mL of buffer and mix thoroughly.
5. Remove cells from incubator and allow the plates to cool to room temperature.
6. Add Caspase-Glo® reagent at a 1:1 ratio (i.e. 100 μL to 100 μL media) and shake at 300 rpm for 30 sec.
7. Incubate cells with Caspase-Glo® reagent for 2 h at room temperature protected from light.
8. Transfer all media to white walled luciferase plates and determined luminescence using the MicroBeta luminometer.
9. Caspase activity is expressed as fold-change relative to the untreated control.

Caspase-Glo® 3/7 reagent is available from Promega (Calif., USA). The Caspase-Glo® reagent contains a DEVD-caspase substrate which is selectively cleaved by caspase enzymes 3 and 7. The resultant molecule then acts as a substrate for a thermostable luciferase enzyme to produce a light signal

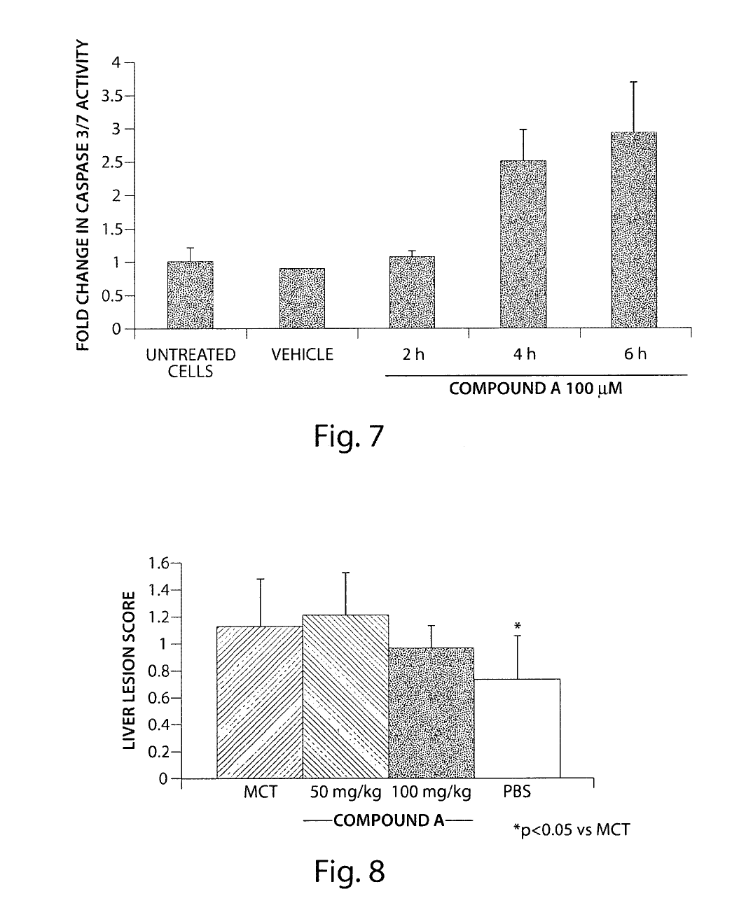

Results: The results of this assay, displayed in FIG. 7, show that compound A increased Caspase activity in a time-dependent fashion.

### Example 4

Compound A was assayed for effects on lung and liver lesions, as well as liver fibrosis in animals suffering from liver fibrosis induced by monocrotaline (MCT).

General Procedure: In this assay, animals were treated with MCT/vehicle, MCT with phosphate-buffered saline (PBS), or MCT with compound A. Following treatment, animals were sacrificed, and lung tissue was perfused and fixed in 10% neutral buffered formalin via the trachea. Lung and liver tissue was removed, sectioned and stained with hematoxylin-eosin. 41 liver and 41 lung slides were obtained for histologic evaluation. All slides were scored blindly without knowledge of experimental conditions. Lung and liver slides were evaluated according to the criteria listed below. Each parameter was subjectively evaluated and scored from 0-5, with 0 being no discernable lesions and 5 indicating the most severe lesions. Final scores for both the liver and lung lesions were calculated by averaging all parameters for each individual tissue. After scoring was complete, the experimental groups were revealed and individual animals were placed in the appropriate groups for statistical and graphical analysis.

### Lung Scoring Criteria and Parameters

1. Alveolar edema
2. Congestion
3. Intra-alveolar hemorrhage
4. Alveolar macrophage infiltrate
5. Degree of erythrophagocytosis
6. Degree of hemosiderosis
7. Thickening of alveolar septa
8. Type II pneumocyte hyperplasia
9. General arterial thickening
10. Thickening of tunica intima
11. Thickening of tunica media
12. Thickening of tunica adventitia
13. Degree of perivascular edema.
14. Perivascular inflammatory cells (lymphs and plasma cells)
15. Increased BALT
16. Endothelial reactivity and prominence.

### Liver Scoring Criteria and Parameters

1. Capsular fibrin
2. Interlobular fibrosis
3. Zone 1, 2, 3 necrosis
4. Central vein, portal vein necrosis
5. Sinusoidal spaces (hepatocytes dropout)
6. Spaces of Disse
7. Bile duct
8. Bile caniliculi
9. Kupffer cells
10. Hepatocyte appositional surfaces
11. Cell and nuclear size variation
12. Cytoplasm variation

Results:

### Description of Lung Lesions

There were three sections of lung on each slide. Sections of lung were obtained from the left lung lobe. The most severe lung lesions were characterized as follows: Moderate to marked numbers of alveolar macrophages infiltrated the parenchyma diffusely. These alveolar macrophages rarely contained intracytoplasmic erythrocytes and hemosiderin. Erythrophagocytosis was a rare to mild feature. Occasionally, aggregates of foamy macrophages were present in alveoli. There was moderate congestion characterized by moderate to marked distention of capillaries and larger blood vessels with sporadic intra-alveolar hemorrhages. There was patchy distribution of marked simultaneous congestion, hemorrhage, erythrophagocytosis and hemosiderosis. More severe lesions exhibited hyaline membrane formation. Moderate type II pneumocyte hyperplasia occurred sporadically, primarily with lesions associated with marked edema, hemorrhage and fibrosis. Pulmonary arteries were moderately to markedly thickened. The thickening primarily involved the tunica media, however the intima and adventitia were moderately affected. There was a mild to moderate inflammatory component composed of lymphocytes, neutrophils, eosinophils and mast cells surrounding vessels. The degree of perivascular edema was mild to marked. Vascular endothelial cells were prominent and nuclei occasionally protruded into vascular lumina. Bronchioles and bronchi rarely had increased peribronchiolar inflammation that included lymphocytes, plasma cells, neutrophils, eosinophils and mast cells. Their lumina were occasionally filled with exfoliated epithelial cells, macrophages, and minimal fibrin and edema. Lymphatic vessels were occasionally dilated and prominent.

### Description of Liver Lesions

There were two sections of liver on each slide. The sections were obtained from the left liver lobe. The predominant finding in the sections of liver was the presence of varying degrees of Zone 1 (periportal) necrosis, which ranged from mild to marked. These findings indicate systemic administration of a toxic substance, which is first distributed to Zone 1, resulting in necrosis of this metabolically active area. Depending on the degree of severity, the necrosis affected Zones 2 and 3 as well. Livers exhibiting massive necrosis also had hepatocyte dropout, with widening and congestion of sinusoids. Moderate numbers of sinusoidal cells with small, condensed nuclei were evident in the experimental groups receiving 50 mg/kg and 100 mg/kg of compound A. These cells may represent apoptotic stellate cells. The presence of neutrophils, lymphocytes or macrophages were not observed. There was evidence of periportal fibrosis in the monocrotaline+vehicle group, which represents an attempt to repair the damaged areas of coagulative necrosis. Some sections in this group had a higher degree of periportal fibrosis than others and also showed signs of hepatocyte regeneration and atypia. The degree of severity for the liver lesions appeared to correlate roughly to the degree of severity of pulmonary lesions.

### Statistical Analysis of Lesion Severity

ANOVA single-factor analysis revealed significant increase in liver lesion severity scores by monocrotaline (MCT) administration in the three experimental groups (Vehicle+MCT, 50 mg/kg compound A+MCT, 100 mg/kg compound A+MCT) compared to Vehicle+PBS control (p values<0.05). The results of this analysis are depicted in FIG. 8.

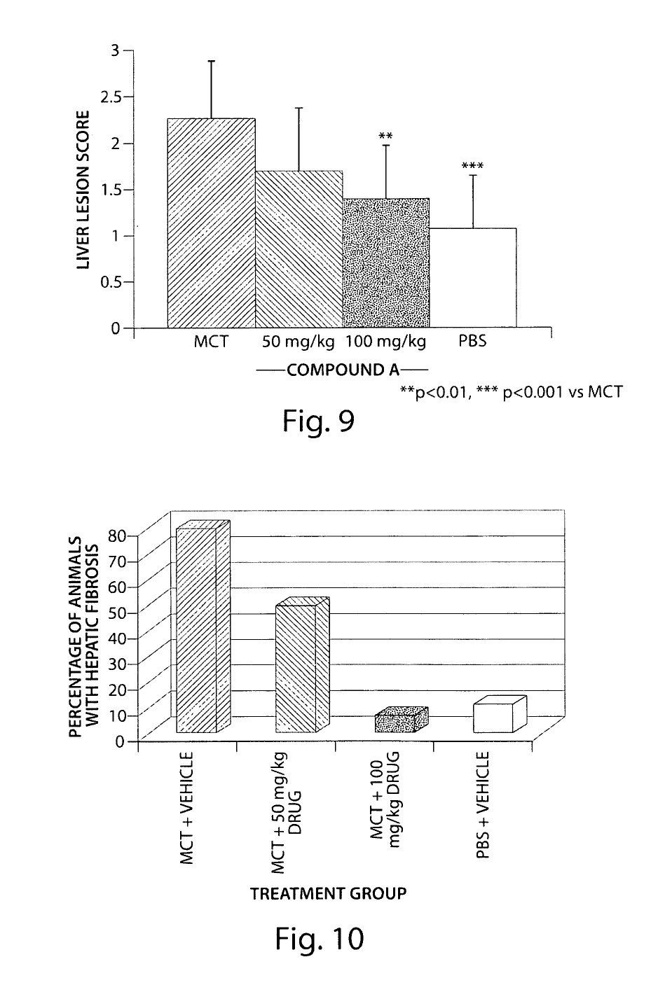

MCT treatment produced significantly higher lung lesion scores indicating greater severity (p<0.001). As depicted in FIG. 9, there was a dose-dependent reduction in lung lesion scores by the administered drug, compound A. A dose of 50 mg/kg of compound A produced a lower lesion score that the Vehicle+MCT group (p=0.09). However, when the dose of compound A was increased to 100 mg/kg, the lung lesions score was reduced in severity and a significant difference was observed (p<0.01). The lungs of the Vehicle+PBS (analogous to normal animal) treated group was similar to the group receiving 100 mg/kg compound A+MCT group, indicating an effective treatment in reducing lung lesions.

### Hepatic Fibrosis Analysis:

There were varying degrees of myofibroblast proliferation and collagen deposition surrounding portal tracts with occasional bridging into zones two and three. Bile duct proliferation was occasionally associated with areas of fibrosis. The degree of periportal fibrosis was evaluated as a separate parameter within each group and the results are as follows:
8 out of 10 (80%) livers treated with monocrotaline+vehicle had mild, moderate or marked fibrosis.
4 out of 8 (50%) livers treated with monocrotaline+50 mg/kg compound A had minimal to mild fibrosis.
1 out of 13 (7%) livers treated with monocrotaline+100 mg/kg compound A had negligible-very minimal (1 small triad) fibrosis.
1 out of 9 (11%) livers treated with vehicle+PBS had minimal fibrosis.

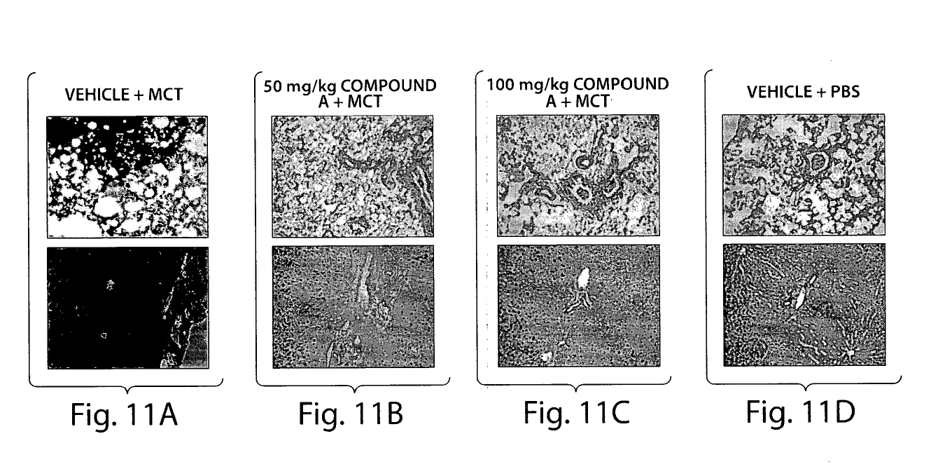

The attenuation of monocrotaline-induced fibrosis by compound A is depicted in FIG. 10. Examples of lung and liver tissue from the various treatment groups are depicted in FIG. 11. The top panels in FIG. 11 show lung tissue, while the bottom panels show liver tissue. Part A in FIG. 11 shows tissue samples from a subject treated with vehicle and MCT, indicating pulmonary hemorrhage and edema with periportal necrosis, hepatocyte dropout and capsular fibrin accululation. Part B in FIG. 11 shows tissue samples from a subject treated with MCT and 50 mg/kg of compound A, indicating improvement of MCT-induced pulmonary lesion with moderate arterial hypertrophy and mild pulmonary edema. Periportal necrosis in Part B appears slightly reduced compared to Part A. Part C in FIG. 11 shows tissue samples from a subject treated with MCT and 100 mg/kg of compound A, which appear to show further reduction of MCT-induced pulmonary lesions with decreased arterial hypertrophy compared to Part B. There appears to be further reduction of periportal necrosis with increased dose of drug. Part D in FIG. 11 shows tissue samples from a subject treated with vehicle and PBS, which appears to show little or no evidence of arterial hypertrophy, pulmonary edema or hemorrhage, and little or no evidence of periportal necrosis.

### Equivalents

Those skilled in the art will recognize, or be able to ascertain using no more than routine experimentation, numerous equivalents to the specific procedures described herein. Such equivalents are considered to be within the scope of the invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the invention and embodiments thereof.

1. A method of treating or preventing fibrosis of an organ of a subject, comprising administering to a subject in need thereof a therapeutically effective amount of a compound of formula I, wherein formula I is represented by: 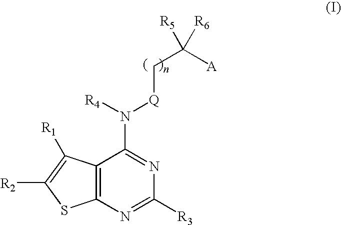
including pharmaceutically acceptable salts, solvates, and/or esters thereof; wherein
R₁ and R₂ represent independently hydrogen, lower alkyl, C₁-C₆ cycloalkyl or cycloheteroalkyl, halogen, halo-substituted alkyl, —COOH, —CN, —NH₂, —NO₂, —OH, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or
R₁ and R₂, taken together with their bonded carbon atoms, form a substituted or unsubstituted C₄-C₇ cycloalkyl or cycloheteroalkyl; wherein the C₄-C₇ cycloheteroalkyl comprises at least one of O, N or S, and the substituted C₄-C₇ cycloalkyl or cycloheteroalkyl comprises at least one substituent selected from halogen, —COOH, —CN, —NH₂, —NO₂, —OH, lower alkyl, substituted lower alkyl, substituted or unsubstituted C₁-C₆ cycloalkyl or cycloheteroalkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, and —R₉-haloalkoxy;
R₃ is H, halogen, —CN, —NH₂, lower alkyl, R₇, —OR₇, —NHR₇, —N(R₇)₂, or substituted or unsubstituted aryl or heteroaryl;
R₄ is H, R₇, or substituted or unsubstituted aryl or heteroaryl;
Q is 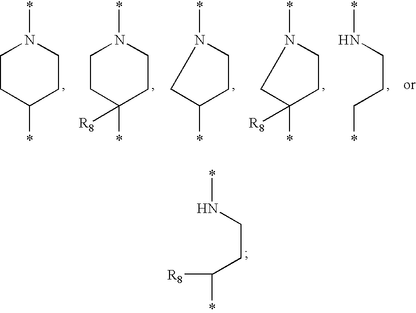
R₅ and R₆ represent independently hydrogen, halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or
R₅, R₆, and A taken together with their bonded carbons, form a substituted or unsubstituted unsaturated 5- or 6-membered carbocyclic ring or a substituted or unsubstituted saturated 5-, 6-, or 7-membered carbocyclic ring, wherein the carbocyclic ring may be a fused biaryl ring or a heterocarbocyclic ring comprising at least one heteroatom selected from the group consisting of O, N, S and P; and the substituted ring comprises at least one of halogen, —COOH, —CN, —NH₂, —NO₂, —OH, lower alkyl, substituted lower alkyl, substituted or unsubstituted aryl or heteroaryl, R₇, —COOR₇, —CONHR₇, —CON(R₇)₂, —OR₇, —NHR₇, —N(R₇)₂, —R₉-alkoxy, —R₉-haloalkyl, or —R₉-haloalkoxy; or R₅, R₆, and A, taken together with their bonded carbons, form an aromatic ring that is optionally substituted on the adjacent carbon atoms to form a bicyclic ring with a 5- or 6-membered unsaturated or saturated ring;
R₇ represents independently for each occurrence substituted or unsubstituted C₁-C₆ alkyl or C₃-C₆ cycloalkyl or C₃-C₆ cycloheteroalkyl;
R₈ is hydrogen, halogen, CN, or a substituted or unsubstituted lower alkyl;
R₉ represents independently for each occurrence substituted or unsubstituted C₁-C₆ alkylene or C₃-C₆ cycloalkylene or C₃-C₆ cycloheteroalkylene;
A is hydrogen or C₁-C₆ alkyl;
n is 0, 1, 2, 3, 4 or 5; and
* represents a point of attachment.

2. The method of claim 1, wherein R₁ and R₂ represent independently hydrogen, lower alkyl, or halogen.

3. The method of claim 1, wherein R₃ and R₄ represent independently hydrogen or unsubstituted C₁-C₆ alkyl.

4. The method of claim 3, wherein Q is

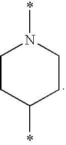

5. The method of claim 3, wherein R₅ is substituted aryl; R₆ is hydrogen; and A is H.

6. The method of claim 1, wherein n is 0 or 1.

7. The method of claim 1, wherein said compound has the following formula: 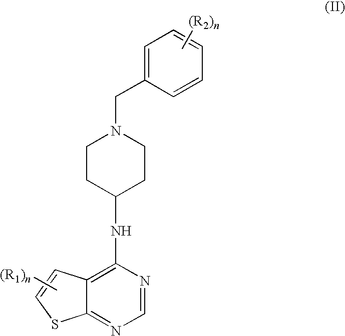
including pharmaceutically acceptable salts, solvates, and/or esters thereof; wherein
R₁ represents independently for each occurrence halogen, lower alkyl, cyano, or trihalomethyl;
R₂ represents independently for each occurrence hydrogen, halogen, cyano, trihalomethyl, lower alkoxy, carboxylate, amide, or a sulfonyl group; and
n represents independently for each occurrence 1 or 2.

8. The method of claim 1, wherein the compound is 5-((4-(6-chlorothieno[2,3-d]pyrimidin-4-ylamino)piperidin-1-yl)methyl)-2-fluorobenzonitrile or a pharmaceutically acceptable salt thereof.

9. The method of claim 1, wherein the compound is 3-((4-(6-chlorothieno[2,3-d]pyrimidin-4-ylamino)piperidin-1-yl)methyl)benzonitrile or a pharmaceutically acceptable salt thereof.

10. The method of claim any one of claims 1, wherein the organ is selected from the group consisting of the liver, the kidney, and the lung.

11-13. (canceled)

14. The method of claim 1, wherein the subject is a human.

15. The method of claim 1, wherein the compound of formula I is administered at a dosage in the range of about 20 mg to about 1000 mg.

16. The method of claim 1, wherein the mode of administration of said compound is oral, intravenous, sublingual, ocular, transdermal, rectal, topical, intramuscular, intra-arterial, subcutaneous, buccal, nasal, or direct delivery to the liver.

17-42. (canceled)

43. A method of treating or preventing fibrosis of an organ of a subject, comprising administering to a subject in need thereof a therapeutically effective amount of 5-((4-(6-chlorothieno[2,3-d]pyrimidin-4-ylamino)piperidin-1-yl)methyl)-2-fluorobenzonitrile, 3-((4-(6-chlorothieno[2,3-d]pyrimidin-4-ylamino)piperidin-1-yl)methyl)benzonitrile, or a pharmaceutically acceptable salt thereof.

44-58. (canceled)

59. The method of claim 1 wherein the fibrosis is associated with hepatitis.

60. The method of claim 59 wherein the hepatitis is hepatitis C.

61. The method of claim 43 wherein the fibrosis is associated with hepatitis.
